(19) United States
(12) Patent Application Publication
(10) Pub. No.: US 2017/0226560 A1
(43) Pub. Date: Aug. 10, 2017

(54) GOLD NANOPARTICLE-BASED HOMOGENEOUS COLORIMETRIC DIAGNOSTIC ASSAY FOR THE DETECTION OF PROTEASES AND PROTEASE INHIBITORS

(71) Applicant: IMRA America, Inc., Ann Arbor, MI (US)

(72) Inventors: Wei Qian, Canton, MI (US); Bing Liu, Ann Arbor, MI (US); Kori Michael Fetters, Ypsilanti, MI (US)

(21) Appl. No.: 15/356,950

(22) Filed: Nov. 21, 2016

Related U.S. Application Data
(63) Continuation of application No. PCT/US2015/031731, filed on May 20, 2015.
(60) Provisional application No. 62/005,175, filed on May 30, 2014.

(51) Int. Cl. C12Q 1/37; G01N 33/58

(52) U.S. Cl. CPC C12Q 1/37; G01N 33/587; G01N 2333/948; G01N 2333/81

(57) ABSTRACT

In the present invention, a method and assay for the detection of proteases and protease inhibitors using colloidal gold nanoparticles and peptide substrates, which are selectively recognized and cleaved by proteases being assayed, is disclosed. In this assay, the mechanism of signal generation relies on peptide sequence induced aggregation of gold nanoparticles, which are used as signal reporters. The peptide sequences that induce aggregation are either the intact peptide substrates or proteolytic fragments of the intact peptide substrate wherein the proteolytic fragments are produced by the protease being assayed. The present invention provides a novel, simple, sensitive, and inexpensive colloidal gold nanoparticle-based colorimetric assay that allows both visual and quantitative detection of proteases and protease inhibitors.

### CROSS-REFERENCE TO RELATED APPLICATION

This application is a continuation of International Application No. PCT/US2015/031731 filed May 20, 2015, which claims the benefit of U.S. Provisional Application No. 62/005,175 filed May 30, 2014, the entire disclosures of these applications being considered part of the disclosure of this application and hereby incorporated by reference.

### STATEMENT REGARDING FEDERALLY SPONSORED RESEARCH

None.

### SEQUENCE LISTING

The Sequence Listing associated with this application is hereby incorporated by reference as found in ASCII text file named SEQUENCELISTING, created on Nov. 21, 2016 and having a size in bytes of 2,952.

### TECHNICAL FIELD

The present invention relates to a method and assay for the detection of proteases and protease inhibitors using colloidal gold nanoparticles and peptide substrates, which are selectively recognized and cleaved by the proteases being assayed. In certain embodiments, the present invention provides a simple, sensitive, and inexpensive colloidal gold nanoparticle-based colorimetric assay that allows both visual and quantitative detection of the activity of proteases and protease inhibitors. In this assay the mechanism of signal generation relies on hydrolytic cleavage of peptide substrates by specific proteases or inhibition of the cleavage by protease inhibitors, which determines the state of aggregation of gold nanoparticles and consequently the color of a solution of colloidal gold nanoparticles.

### BACKGROUND

Proteases are a class of enzymes that hydrolytically cleave peptide sequences and proteins at specific sites within peptide sequences or they remove amino acids from the ends of peptide sequences. Proteases are estimated to comprise 2% of the human genome and control a diverse array of biological processes in living organisms by playing pivotal roles in protein activation, cell regulation and signaling, as well as in the generation of amino acids for protein synthesis or utilization in other metabolic pathways.

Therefore, it is not surprising that perturbations of protease activity are associated with multiple pathological conditions such as cancer, neurodegenerative disorders, inflammation and cardiovascular diseases. Altered protease activity could be indicative of cancer prognosis and therefore could validate the use of a specific protease as a cancer biomarker. For example, prostate-specific antigen (PSA), a serine protease, is best known as a prostate cancer biomarker and its level in a man is used as one form of early detection of possible prostate cancer.

Because proteases are of great relevance to biology, medicine, and biotechnology, sensitive assays capable of detecting proteolytic activities of proteases will be extremely valuable and have broad applications in drug screening, diagnosis, and the development of effective and selective therapeutics for a wide variety of applications.

In the past several decades, a number of assay methods, such as, enzyme-linked immunosorbent assays (ELISA), radioisotope-based assays, and fluorogenic substrate-based assays, have been developed and used for the detection of protease activity. However, efforts to increase the utility and applicability of these assays for the detection of protease activity are frustrated by the limitations of potential assay safety issues associated with use of radioisotopes, assay sensitivity, assay specificity, requirements for specific instruments, and because they can be laboriously time-consuming. Therefore, there is an urgent demand for simple, low-cost, highly sensitive assays capable of on-site detection of protease activity. To address this urgent demand, we disclose in the present invention a novel design of a colloidal gold nanoparticle-based colorimetric assay for the detection of a variety of protease activities.

Colloidal gold nanoparticles are gold nanoparticles dispersed in a dispersion medium, typically water, but other media can also be used as discussed below. Gold nanoparticles have attracted substantial interest from scientists for over a century because of their unique physical, chemical, and surface properties, such as: (i) size- and shape-dependent strong optical extinction and scattering which is tunable from ultraviolet (UV) wavelengths all the way to near infrared (NIR) wavelengths; (ii) large surface areas for conjugation to functional ligands; and (iii) little or no long-term toxicity or other adverse effects in vivo allowing their high acceptance level in living systems. Colloidal gold nanoparticles, also referred to as gold nanocolloids, are now being investigated for their potential use in a wide variety of biological and medical applications as imaging contrast agents (Nat. Biotechnol. 2008, 26, 83 and Nano Lett. 2005, 5, 829), therapeutic agents (Nano Lett. 2007, 7, 1929 and Sci. Transl. Med. 2010, 2), biological sensors (Chem. Soc. Rev. 2008, 37, 2028), and cell-targeting vectors (Nano Lett. 2007, 7, 247).

Currently, the overwhelming majority of gold nanocolloids are prepared by using the standard wet chemical sodium citrate reduction of chloroauric acid (HAuCl4) methodology. This method results in the synthesis of spherical gold nanoparticles with diameters ranging from 5 to 200 nanometers (nm) which are capped or covered with negatively charged citrate ions. The citrate ion capping creates a negative surface charge on the nanoparticles which prevents the nanoparticles from aggregating by providing electrostatic repulsion between nanoparticles.

Other wet chemical methods for formation of colloidal gold nanoparticles include the Brust method, the Perrault method and the Martin method. The Brust method relies on reaction of chloroauric acid with tetraoctylammonium bromide in toluene and sodium borohydride. The Perrault method uses hydroquinone to reduce the HAuCl4 in a solution containing gold nanoparticle seeds. The Martin method uses reduction of HAuCl4 in water by NaBH4 wherein the stabilizing agents HCl and NaOH are present in a precise ratio. All of the wet chemical methods rely on first converting gold (Au) with a strong acid into the atomic formula HAuCl4 and then using this atomic form to build up the nanoparticles in a bottom-up type of process. All of the methods require the presence of stabilizing agents to prevent the gold nanoparticles from aggregating and precipitating out of solution.

In addition to the wet chemical methods, several physical methods exist for making metal nanoparticles. One of these physical methods of making metal nanoparticles is based on pulsed laser ablation of a metal target immersed in a liquid, and it has been attracting increasingly widespread interest. In contrast to the chemical procedures, pulsed laser ablation of a metal target immersed in a liquid offers the possibility of generating stable nanocolloids while avoiding chemical precursors, reducing agents, and stabilizing ligands, all of which could be problematic for the subsequent functionalization and stabilization of the metal nanoparticles. Therefore, since it was pioneered by Henglein and Fojtik for preparing nano-size particles in either organic solvents or aqueous solutions as well as by Cotton for preparation of water-borne surface-enhanced Raman scattering active metallic nanoparticles with bare surfaces in 1993, the application of pulsed laser ablation of metal targets in liquids has gained much interest. In the initial procedures the lasers utilized were nanosecond lasers. With the advent of femtosecond lasers, which are capable of eliminating some problems associated with the use of nanosecond lasers, interest has picked up in these methods. Compared to laser ablation with nanosecond pulses, the irradiation of metal targets by femtosecond laser pulses offers a precise laser-induced breakdown threshold and can effectively minimize the heat affected zones since the femtosecond laser pulses release energy to electrons in the target on a time-scale much faster than electron-phonon thermalization processes. Characterized by its simplicity of procedure, versatility with respect to metals or solvents, and nanoparticle growth in a controllable, contamination-free environment, pulsed laser-induced ablation from solid targets has evolved as one of the most important physical methods for obtaining colloidal metallic nanoparticles.

Among the unique optical and electronic properties of gold nanocolloids mentioned above, their localized surface plasmon resonance (LSPR) has received particular interest. The physical origin of the LSPR is associated with coherent oscillations of conduction-band electrons on the gold nanoparticle surface upon interaction with light with the exact LSPR band being extremely sensitive to the size, shape, and aggregation state of the nanoparticles; to the dielectric properties of the surrounding medium; and to the adsorption of ions on the surface of nanoparticles. For example, for gold (Au) nanocolloids with an average particle diameter of 20 nanometers (nm), the maximum absorbance of the localized surface plasmon resonance of this Au nanocolloid is at 520 nm±5 nm. Upon aggregation of the gold nanoparticles, the localized surface plasmon resonance of this Au nanocolloid with an average particle diameter of 20 nm is significantly modified with a decrease of the absorption band at 520 nm±5 nm and appearance of a new absorption band maximum at longer wavelengths of between 600 to 700 nm due to dipole coupling between the localized surface plasmon resonance (LSPR) of neighboring particles forming the aggregates, thus leading to color changes of the solution from an initial color of a pink-red to a violet-blue color. These color changes are visible to the naked eye and can provide a qualitative measure of the degree of aggregation of the gold nanoparticles.

Taking advantage of the color changes of a gold nanocolloid solution from pink-red to violet-blue arising from the inter-particle localized surface plasmon resonance (LSPR) coupling during gold nanoparticle aggregation, an absorption-based colorimetric assay with gold nanoparticles as signal reporters has been designed in the present invention for the detection of a target analyte or a biological process that is able to directly or indirectly trigger gold nanoparticle aggregation. As gold nanoparticles have extremely high extinction coefficients, for example, 8.8×108 M-1 cm-1 at 520 nm±5 nm for 20 nm spherical gold nanoparticles which is more than 1000 times higher than those of organic dyes, gold nanoparticle-based colorimetric assays will have high sensitivity, much higher than those of conventional bio detection assays that use a fluorescence signal. In addition, the color change of a gold nanocolloid solution from pink-red to violet-blue upon aggregation of the gold nanoparticles could be observed by the naked eye and therefore, sophisticated instruments are not required for qualitative analysis using gold nanoparticle-based colorimetric assays. For quantitative analysis using gold nanoparticle-based colorimetric assays, the absorption spectra of gold nanocolloids can be recorded using a standard spectrophotometer or plate reader. The ratio of the absorbance at 520 nm±5 nm, which corresponds to dispersed gold nanoparticles, to the absorbance at a longer wavelength (between 600 to 700 nm), which corresponds to aggregated gold nanoparticles, is often used to quantify the aggregation process or color change. Sometimes, an aggregation parameter, which measures the variation of the integrated absorbance between, for example, 600 and 700 nm, is used for quantitative analysis and this method can provide a higher sensitivity.

Due to their simplicity and high sensitivity, gold nanoparticle-based colorimetric assays have attracted an increasing level of attention in broad applications, such as clinical diagnostics, drug discovery, drug screening, and environmental contaminant analysis. Since the first gold nanoparticle-based colorimetric assay was developed by Chad Mirkin and co-workers for the detection of DNA (Science 1997, 277, 1078), this platform has been increasingly applied for the detection of a large variety of target analytes, including proteins, small molecules, metal ions, and even cells and is quickly becoming an important alternative to conventional detection techniques.

In the present invention, we have developed methods and assays for the detection of proteases and protease inhibitors with high sensitivity using gold nanocolloids and peptide substrates based on unique mechanisms of peptide substrate induced aggregation or disaggregation of gold nanoparticles. The application of gold nanoparticles for the detection of proteases utilizing a peptide substrate induced aggregation of gold nanoparticles has been reported by Paolo Scrimin and co-workers (Proc. Natl. Acad. Sci. USA 2006, 103, 3978). In their process, the peptide substrate used in the detection has to contain two cysteines with one cysteine located at the amino-terminus of the peptide substrate and the other cysteine located at the carboxyl-terminus of the peptide substrate. Cysteine binds to gold nanoparticles via a sulfur-gold bond, so the intact peptide substrate featuring two cysteines crosslinks the gold nanoparticles through the two cysteine arms, which results in the color of the solution of colloidal gold nanoparticles turning from pink-red to violet-blue due to aggregation of the gold nanoparticles by the intact peptide substrate. Conversely, after cleavage of the intact peptide substrate by a specific protease, the proteolyzed peptide fragments containing only one cysteine located at either the amino-terminus or the carboxyl-terminus would not cross-link gold nanoparticles, resulting in no colorimetric change. Thus in the process of Paolo Scrimin and co-workers, protease activity could be determined by measuring the reduction of the aggregation of gold nanoparticles by the presence of the protease of interest. However, in our studies we have surprisingly found that peptide substrates containing only one cysteine, which can located at the amino-terminus, the carboxyl-terminus, or internally in the peptide substrate in some examples, could also be used to induce aggregation of gold nanoparticles under certain circumstances. By taking advantage of this phenomenon, we describe in the present invention a new design of simple, sensitive, and inexpensive colloidal gold nanoparticle-based colorimetric assays that allow both visual and quantitative detection of proteases and protease inhibitors.

### SUMMARY OF THE INVENTION

The present invention relates to methods and assays for the detection of proteases and protease inhibitors using gold nanoparticles and peptide substrates, which are selectively recognized and cleaved by the proteases that are either being assayed for or being used for the detection of their corresponding protease inhibitors. The peptide substrates are designed to include: amino acid sequences recognized by and cleaved by proteases of interest; at least one functional group that can bind to gold nanoparticles and that is located on the carboxyl terminus, on the amino terminus, or internally in which case it is exposed by the hydrolytic cleavage; and the property that the intact peptide substrate or at least one of its cleavage products causes aggregation of the gold nanoparticles. These unique peptide substrates can be used to detect and quantify both the proteases of interest and specific inhibitors of these proteases. The gold nanoparticles used can either have a negative or a positive surface charge prior to interaction with the peptide substrates.

In some embodiments, the present invention provides methods for detecting the amount of a protease that rely on the ability of an intact peptide substrate to cause aggregation of the gold nanoparticles. In this embodiment, the intact peptide substrate comprises at least one first functional group having an affinity for the gold nanoparticles, preferably the first functional group can form a covalent bond with the gold nanoparticles; at least one second functional group that can form an ionic bond with the nanoparticles thereby providing the peptide substrate with the ability to cause aggregation of the gold nanoparticles when fully intact; and at least one amino acid sequence that will be selectively recognized and cleaved by the protease being assayed and located between the first and the second functional groups, so the peptide substrate cannot aggregate the nanoparticles after cleavage by the protease. The gold nanoparticles can either have a positive or a negative surface charge, which determines in part the amino acids required in the peptide substrate to form the second functional group. In a first step a liquid, which can be a biological fluid or other liquid, suspected of containing the protease is mixed with a solution containing a known amount of the peptide substrate to generate a mixture. In a second step the mixture is incubated at a predetermined temperature for a sufficient amount of time to enable some hydrolytic cleavage of the peptide substrate by the protease. Generally the predetermined temperature is at least 20° C.; however the present invention can also be used to detect both high temperature tolerant and cold tolerant proteases so the reaction temperatures may be adjusted as required by the protease being studied. Generally the incubation time will range from 10 minutes to three hours; however other times may be required depending on the incubation temperature and the amount of protease being assayed. In a third step, after the hydrolytic cleavage incubation step a colloidal suspension of gold nanoparticles with a pink-red color, meaning non-aggregated nanoparticles, is added to the mixture so that nanoparticle aggregation is induced by the remaining intact peptide substrate resulting in the formation of a final solution with a characteristic color shift from the initial pink-red to a violet-blue in 30 minutes or less. In a forth step qualitatively detecting the amount of said protease in the liquid by comparing the color of the final solution with colors of standard samples containing known amounts of the peptide substrate and gold nanoparticles. In addition, quantitatively determining the amount of the protease in the liquid by comparing the absorbance of the final solution measured using an UV-Vis spectrophotometer or plate reader with the absorbance of a standard curve prepared using samples containing known amounts of the peptide substrate and gold nanoparticles.

In some embodiments, the present invention provides methods for detecting the amount of a protease inhibitor in a sample that rely on the ability of an intact peptide substrate to cause aggregation of the gold nanoparticles using a process similar to that described above for detection of a protease. The gold nanoparticles used can have either a positive or a negative surface charge, which determines in part the amino acids required in the peptide substrate. The first step is identifying an appropriate protease, whose hydrolytic cleavage of a peptide substrate will be blocked by the protease inhibitor being assayed. As described above, the intact peptide substrate comprises at least one functional group having an affinity for the gold nanoparticles; at least one amino acid sequence that will be selectively recognized and cleaved by the protease; and the ability through a second functional group to cause aggregation of the gold nanoparticles when fully intact but not after cleavage by the protease. The second step includes mixing a liquid, which may be a biological fluid, suspected of containing the protease inhibitor with a solution having a known amount of the protease and incubating the solution at a predetermined temperature, as noted above generally a temperature of above about 20 degrees Celsius, for a sufficient amount of time to enable said protease inhibitor to block the function of the protease regarding hydrolytic cleavage of the peptide substrate. The third step involves adding an aqueous solution of the peptide substrate specific for the protease to the solution of suspected protease inhibitor and protease to generate a mixture containing the protease, the suspected protease inhibitor, and the peptide substrate and incubating the mixture at a predetermined temperature, as discussed above generally a temperature of above about 20 degrees Celsius, for a sufficient amount of time to enable some hydrolytic cleavage of the peptide substrate by the protease. The forth step includes adding a colloidal suspension of gold nanoparticles with a pink-red color, meaning non-aggregated nanoparticles, to the mixture so that nanoparticle aggregation is induced by the remaining intact peptide substrate resulting in the formation of a final solution with a characteristic color shift in 30 minutes or less. The fifth step involves qualitatively determining the amount of the protease inhibitor in the liquid by comparing the color of the final solution with colors of standard samples containing known amounts of the protease inhibitor, protease, peptide substrate and gold nanoparticles. In addition, quantitatively determining the amount of the protease inhibitor in the liquid by comparing the light absorbance of the final solution measured using an UV-Vis spectrophotometer or plate reader with the absorbance of a standard curve prepared using samples containing known amounts of the protease inhibitor, protease, peptide substrate and gold nanoparticles. As can be understood from the above disclosure of the present inventive assay methods, this assay procedure can also be used to detect previously unknown proteases or protease inhibitors. Detection of an unknown protease can be accomplished by comparing the degree of aggregation in the presence and absence of a series of dilutions of a sample suspected to contain a protease. In a similar fashion, the effect of a series of dilutions of a suspected protease inhibitor on the aggregation state caused by a series of protease dilutions can be determined to discover previously unknown protease inhibitor activity.

In some embodiments, the present invention provides methods for detecting a protease that rely on the inability of an intact peptide substrate to cause aggregation of the gold nanoparticles and the ability of the cleaved peptide substrate to cause aggregation of the gold nanoparticles. In this embodiment the intact peptide substrate comprises at least one first functional group having affinity for the gold nanoparticles, preferably capable of forming a covalent bond with the gold nanoparticle; at least one amino acid sequence that will be selectively recognized and cleaved by the protease being assayed; and at least a second functional group capable of forming an ionic bond with the gold nanoparticles and located such that the cleavage sit is outside the sequence between the first and second functional groups. The second functional is blocked from interacting with the gold nanoparticles in the intact peptide substrate but exposed after cleavage, hence the inability of the intact peptide to cause aggregation of the gold nanoparticles when fully intact but the ability to cause aggregation of the gold nanoparticles after cleavage by the protease. The second functional group can be “blocked” either due to a structural interference in the intact peptide sequence or simply because it is not accessible to interact with the gold nanoparticle by virtue of its interior location. The gold nanoparticles can either have a positive or a negative surface charge, which determines in part the amino acids required in the peptide substrate. In a first step a liquid, which can be a biological fluid or other liquid, suspected of containing the protease is mixed with a solution containing a known amount of the peptide substrate to generate a mixture. In a second step the mixture is incubated at a predetermined temperature, generally above about 20 degrees Celsius as noted above, for a sufficient amount of time to enable some peptide substrate cleavage. In a third step a colloidal suspension of gold nanoparticles with a pink-red color, meaning non-aggregated nanoparticles, is added to the solution so that nanoparticles can be aggregated by one or more of the peptide cleavage products resulting in a final solution with a characteristic color shift from pink-red to violet-blue in 30 minutes or less. Qualitatively determining the amount of the protease in the liquid by comparing the color of the final solution with colors of standard samples containing known amounts of the protease, peptide substrate and gold nanoparticles. Alternatively, the standards can be prepared using the gold nanoparticles and known amounts of the aggregating hydrolytic fragment. In addition, quantitatively determining the amount of the protease in the liquid by comparing the light absorbance of the final solution measured using an UV-Vis spectrophotometer or plate reader with the absorbance of a standard curve prepared using samples containing known amounts of the protease, peptide substrate and gold nanoparticles. The standards can also be prepared using the gold nanoparticles and aggregating hydrolytic fragment as described above. In this embodiment the first functional group that binds to the gold nanoparticles can either be exposed in the intact peptide substrate, such as on the amino or carboxyl terminal ends, or it can be exposed following the hydrolytic cleavage. Likewise, the second functional group can either be exposed or blocked in the intact peptide with it being exposed for sure in a hydrolytic fragment. Thus, in one embodiment both the first and second functional groups are not available in the intact peptide and both are available in one of the hydrolytic fragments; or one of the first or the second functional groups are not available in the intact peptide substrate but is exposed with the other of the first and second functional groups in one of the hydrolytic fragments. For example, one can envision a cleavage product that has an amino or carboxyl terminal cysteine which was located internally in the intact peptide and thus unable to bind to the nanoparticles in the intact peptide substrate.

In some embodiments, the present invention provides methods for detecting the amount of a protease inhibitor in a sample that rely on the inability of an intact peptide substrate to cause aggregation of the gold nanoparticles using a process similar to that described above for detection of a protease. The gold nanoparticles used can have either a positive or a negative surface charge, which determines in part the amino acids required in the peptide substrate. The first step is identifying an appropriate protease, whose hydrolytic cleavage of a peptide substrate will be blocked by the protease inhibitor being assayed. The intact peptide substrate comprises at least one first functional group having an affinity for the gold nanoparticles, preferably group that forms a covalent bond with gold nanoparticles; at least one amino acid sequence that will be selectively recognized and cleaved by the protease; and the inability to cause aggregation of the gold nanoparticles when fully intact but the ability after cleavage by the protease to cause aggregation of the gold nanoparticles. The location of the second functional group and its properties are as described above for the embodiment wherein this type of peptide substrate is used to detect a protease. The second step includes mixing a liquid, which may be a biological fluid or other fluid, suspected of containing the protease inhibitor with a solution having a known amount of the protease and incubating the solution at a predetermined temperature, generally of above about 20 degrees Celsius, for a sufficient amount of time to enable said protease inhibitor to block the function of the protease regarding hydrolytic cleavage of the peptide substrate. The third step involves adding an aqueous solution of the peptide substrate specific for the protease to the solution of suspected protease inhibitor and protease to generate a mixture containing the protease, the suspected protease inhibitor, and the peptide substrate and incubating the mixture at a predetermined temperature, generally of above about 20 degrees Celsius, for a sufficient amount of time to enable some hydrolytic cleavage of the peptide substrate by the protease. The forth step includes adding a colloidal suspension of gold nanoparticles with a pink-red color, meaning non-aggregated nanoparticles, to the mixture so that nanoparticle aggregation is induced by the cleaved peptide substrate resulting in the formation of a final solution with a characteristic color shift in 30 minutes or less. The fifth step involves qualitatively determining the amount of the protease inhibitor in the liquid by comparing the color of the final solution with colors of standard samples containing known amounts of the protease inhibitor, protease, peptide substrate and gold nanoparticles. Alternatively as described above the standards can be prepared with just the gold nanoparticles and known amounts of the aggregating peptide fragment. In addition, quantitatively determining the amount of the protease inhibitor in the liquid by comparing the light absorbance of the final solution measured using an UV-Vis spectrophotometer or plate reader with the absorbance of a standard curve prepared using samples containing known amounts of the protease inhibitor, protease, peptide substrate and gold nanoparticles.

In some embodiments, the present invention is directed to an assay system for detecting an amount of a protease comprising: colloidal gold nanoparticles; an effective peptide substrate having at least one first functional group with an affinity for the gold nanoparticles, an amino acid sequence that will be selectively recognized and cleaved by the protease being assayed, and the ability through a second functional group to cause aggregation of the gold nanoparticles either as the intact peptide substrate or as the cleaved peptide substrate; and standard samples containing known amounts of the protease and peptide substrate being assayed to allow for creation of a standard curve. Alternatively the standard can be prepared using the gold nanoparticles and known amounts of the aggregating peptide sequence. The assay system may also optionally include liquids for dissolving the peptide substrate and the standard samples, and appropriate reaction containers. In some embodiments the assay system may include a color card showing colors and the associated degree of aggregation of the gold nanoparticles to allow for a rapid visual estimate of the amount of protease.

In some embodiments, the present invention is directed to an assay system for detecting an amount of a protease inhibitor comprising: colloidal gold nanoparticles; an appropriate protease, meaning one who's cleavage of the peptide substrate will be blocked by the protease inhibitor; an effective peptide substrate for the protease having at least one first functional group with an affinity for the gold nanoparticles, an amino acid sequence that will be recognized and cleaved by the protease, and the ability through a second functional group to cause aggregation of the gold nanoparticles either as the intact peptide or as the cleaved peptide substrate; and standard samples containing known amounts of the protease inhibitor, the protease, and the peptide substrate to allow for creation of a standard curve. The assay system may optionally include liquids for dissolving the protease, the peptide substrate, and the standard samples; and appropriate reaction containers. In some embodiments the assay system may include a color card showing colors and the associated degree of aggregation of the gold nanoparticles to allow for a rapid visual estimate of the amount of protease inhibitor.

### BRIEF DESCRIPTION OF THE DRAWINGS

FIGS. 1a to 1d schematically illustrate several embodiments of the colloidal gold nanoparticle-based colorimetric assay according to the present invention as used to detect the activity of a protease, such as trypsin;

FIG. 2 schematically illustrates a laser-based ablation system for the top-down production of colloidal gold nanoparticles in a liquid in accordance with the present invention;

FIG. 3 illustrates the UV-VIS absorption spectrum of stable colloidal gold nanoparticles prepared according to the present invention by laser ablation of a bulk gold target in deionized water and a transmission electron microscopy (TEM) picture of these stable colloidal gold nanoparticles with an average particle diameter of 20 nanometers is shown in the inset;

FIGS. 4a to 4c display the induced aggregation of colloidal gold nanoparticles with an average diameter of 20 nm by additions of solutions of a peptide substrate: (a) is a series pf photographs showing generation of aggregated colloidal gold nanoparticle solutions with different colors depending on the concentration of a peptide substrate with an amino acid sequence of SEQ ID NO: 1 Lys-Lys-Gly-Phe-Pro-Arg-Gly-Gly-Asp-Cys being added to solutions, the tonality of the solution of colloidal gold nanoparticles changes from a pink-red when no aggregation is present to a violet-blue with increasing the concentrations of the peptide added to the solution; (b) Ultraviolet-visible spectra of the solutions of colloidal gold nanoparticles in the presence of various concentration of the peptide substrate SEQ ID NO: 1 Lys-Lys-Gly-Phe-Pro-Arg-Gly-Gly-Asp-Cys; and (c) Linear dependence of the ratio of the absorbance at 650 nm to the absorbance at 520 nm of the solution of colloidal gold nanoparticles on the concentration of the peptide substrate SEQ ID NO: 1 Lys-Lys-Gly-Phe-Pro-Arg-Gly-Gly-Asp-Cys added to a solution of colloidal gold nanoparticles in a range from 500 nM to 1000 nM of peptide substrate;

FIGS. 5a and b display the dependence of the peptide-induced aggregation of the colloidal gold nanoparticles on the time period over which the peptide substrate SEQ ID NO: 1 Lys-Lys-Gly-Phe-Pro-Arg-Gly-Gly-Asp-Cys was pre-exposed to the protease trypsin: (a) Ultraviolet-visible spectra of the solutions of colloidal gold nanoparticles after being added to 1000 nM solutions of the peptide substrate SEQ ID NO: 1 Lys-Lys-Gly-Phe-Pro-Arg-Gly-Gly-Asp-Cys pre-exposed to trypsin at a concentration of 200 pM for 0 min (□), 15 min (◯), 30 min (⋄), and 60 min (⋆); (b) The ratio of the absorbance at 650 nm to the absorbance at 520 nm of the solution of colloidal gold nanoparticles after being added to the solutions of the peptide substrate SEQ ID NO: 1 Lys-Lys-Gly-Phe-Pro-Arg-Gly-Gly-Asp-Cys as a function of the pre-exposure time period;

FIGS. 6a and b display the dependence of the aggregation of the colloidal gold nanoparticle-based colorimetric assay on the level of the protease trypsin: (a) Ultraviolet-visible spectra of the solutions of colloidal gold nanoparticles after being added to solutions of the peptide substrate SEQ ID NO: 1 Lys-Lys-Gly-Phe-Pro-Arg-Gly-Gly-Asp-Cys pre-exposed to trypsin at different concentrations up to 2 nM for 60 minutes; (b) The ratio of the absorbance at 650 nm to the absorbance at 520 nm of the solution of colloidal gold nanoparticles after being added to the solutions of the peptide substrate SEQ ID NO: 1 Lys-Lys-Gly-Phe-Pro-Arg-Gly-Gly-Asp-Cys pre-exposed to trypsin for 60 min as a function of the concentration of the trypsin;

FIGS. 7a and b illustrate that the limit of the detection of trypsin using the gold nanoparticle-based colorimetric assay could be improved by increasing the time period over which the peptide substrate SEQ ID NO: 1 Lys-Lys-Gly-Phe-Pro-Arg-Gly-Gly-Asp-Cys was pre-exposed to trypsin; (a) Ultraviolet-visible spectra of the solutions of colloidal gold nanoparticles after being added to solutions of the peptide substrate SEQ ID NO: 1 Lys-Lys-Gly-Phe-Pro-Arg-Gly-Gly-Asp-Cys pre-exposed to trypsin at different concentrations up to 200 pM for 3 hours; (b) The ratio of the absorbance at 650 nm to the absorbance at 520 nm of the solution of colloidal gold nanoparticles after being added to solutions of the peptide substrate SEQ ID NO: 1 Lys-Lys-Gly-Phe-Pro-Arg-Gly-Gly-Asp-Cys pre-exposed to trypsin for 3 hours as a function of the concentration of trypsin;

FIGS. 8a and b illustrate that limit of the detection of trypsin using the gold nanoparticle-based colorimetric assay could be further improved by using gold nanoparticles with a larger average diameter: (a) Ultraviolet-visible spectra of solutions of colloidal gold nanoparticles having an average diameter of 30 nm after being added to solutions of the peptide substrate SEQ ID NO: 1 Lys-Lys-Gly-Phe-Pro-Arg-Gly-Gly-Asp-Cys pre-exposed to trypsin at different concentrations up to 200 pM for 3 hours; (b) The ratio of the absorbance at 650 nm to the absorbance at 520 nm of the solution of 30 nm colloidal gold nanoparticles after being added to the solutions of peptide substrate SEQ ID NO: 1 Lys-Lys-Gly-Phe-Pro-Arg-Gly-Gly-Asp-Cys pre-exposed to trypsin for 3 hours as a function of the concentration of trypsin;

FIGS. 9a to 9c illustrate use of the present invention in an assay of the protease Protease K using 20 nm gold nanoparticles having a negative surface charge, (a) illustrates the scheme of the assay, (b) shows the Ultraviolet-visible spectra of colloidal gold nanoparticles after being added to solutions of the peptide substrate shown in 9(a) at 750 nM pre-exposed to different concentrations of Protease K up to 300 pM for 60 minutes at 20° C., (c) is a graph of the ratio of the absorbance at 700 nm to the absorbance at 520 nm of the solution of gold nanoparticles after being added to the solutions of the peptide substrate pre-exposed to Protease K for 60 minutes as a function of the concentration of Protease K;

FIGS. 10a to 10c illustrate use of the present invention in an assay of the protease Thrombin using 20 nm gold nanoparticles having a negative surface charge, (a) illustrates the scheme of the assay, (b) shows the Ultraviolet-visible spectra of colloidal gold nanoparticles after being added to solutions of the peptide substrate shown in 10(a) at 500 nM pre-exposed to different concentrations of Thrombin up to 30 nM for 60 minutes at 20° C., (c) is a graph of the ratio of the absorbance at 620 nm to the absorbance at 520 nm of the solution of gold nanoparticles after being added to the solutions of the peptide substrate pre-exposed to Thrombin for 60 minutes as a function of the concentration of Thrombin; and

FIGS. 11a to 11d illustrate use of the present invention in an assay of the protease Trypsin using 20 nm gold nanoparticles having a negative surface charge, (a) illustrates the scheme of the assay wherein the intact peptide substrate does not aggregate the gold nanoparticles and a hydrolytic fragment does aggregate the gold nanoparticles, (b) shows the Ultraviolet-visible spectra of colloidal gold nanoparticles after being added to solutions of the peptide substrate shown in 11(a) at 600 nM pre-exposed to different concentrations of Trypsin up to 500 pM for 60 minutes at 20° C., (c) is a graph of the ratio of the absorbance at 610 nm to the absorbance at 520 nm of the solution of gold nanoparticles after being added to the solutions of the peptide substrate pre-exposed to Trypsin for 60 minutes as a function of the concentration of Thrombin, and (d) is a series of photographs of solutions showing the color change as the nanoparticles aggregate in increasing levels of the hydrolytic fragments.

### DETAILED DESCRIPTION

Proteases are a class of enzymes that cleave other proteins at specific sites within peptide recognition sequences, or remove amino acids from the ends of peptide sequences. The recognition sequence for a protease can be a single amino acid or it can be a sequence of amino acids. Many proteases are capable of recognizing several amino acid sequences and thus can cut a peptide bond next to one of several amino acids; others can be very specific and require a longer recognition sequence to maintain enzymatic specificity. For example, digestive enzymes like trypsin can act on a wide variety of protein substrates since it cuts on the carboxyl side of all arginine or lysines in an amino acid sequence unless they are bound to a C-terminal proline. On the other hand, the proteases involved in blood clotting have very specific and long recognition sequences to maintain their specificity. In the present specification and claims it is to be understood that the recognition sequence for a protease may comprise several structurally related sequences and that the cleavage of the peptide may occur at an amino acid adjacent to or within the sequence depending on the protease. Proteases are estimated to comprise 2% of the human genome and control a diverse array of biological processes in living organisms by playing pivotal roles in protein activation, cell regulation and signaling, as well as in the generation of amino acids for protein synthesis or utilization in other metabolic pathways. There are currently six broadly defined groups of proteases: serine proteases, threonine proteases, cysteine proteases, aspartate proteases, glutamic acid proteases, and metalloproteases. Within each group there are hundreds of proteases grouped by their structure, mechanism and catalytic residue order and sequence similarity. Because of their great relevance to biology, medicine, and biotechnology, a sensitive assay method capable of detecting a variety of proteases will be extremely valuable and have broad applications in drug screening, diagnosis, and the development of effective and selective therapeutics. Several current standard assays, including enzyme-linked immunosorbent assays, radioisotope-based assays, and fluorogenic substrate-based assays, can suffer from radioisotope safety issues, lack of sufficient sensitivity, lack of the ability for the specific detection, and they can be laboriously time-consuming. Thus, there is an urgent demand for simple, low-cost, highly sensitive assays capable of on-site detection of protease activity with high specificity.

Because of their unique optical and electronic properties, such as size and shape-dependent localized surface plasmon resonance (LSPR), which is tunable from ultraviolet (UV) wavelengths all the way to near infrared (NIR) wavelengths, gold nanoparticles have attracted substantial interest from scientists for over a century and are now being widely investigated for their potential use in a variety of biological and medical applications, including as imaging contrast agents (Nat. Biotechnol. 2008, 26, 83 and Nano Lett. 2005, 5, 829), therapeutic agents (Nano Lett. 2007, 7, 1929 and Sci. Transl. Med. 2010, 2), biological sensors (Chem. Soc. Rev. 2008, 37, 2028), and cell-targeting vectors (Nano Lett. 2007, 7, 247).

The physical origin of the LSPR is associated with coherent oscillations of conduction-band electrons on the gold nanoparticle surface upon interaction with light with the exact LSPR band being extremely sensitive to not only the size and shape of gold nanoparticles but also the aggregation state of the gold nanoparticles. For example, after aggregation, the localized surface plasmon resonance of a gold (Au) nanocolloid with an average particle diameter of 20 nm is significantly modified with a decrease of the absorption band at 520 nm±5 nm and an appearance of new absorption band at longer wavelengths between 600 to 700 nm due to dipole coupling between the localized surface plasmon resonance (LSPR) of neighboring particles forming the aggregates, thus leading to color changes of the solution from pink-red to violet-blue. Taking advantage of the color changes of a gold nanocolloid solution from pink-red to violet-blue arising from the inter-particle localized surface plasmon resonance (LSPR) coupling during gold nanoparticle aggregation, an absorption-based colorimetric assay with gold nanoparticles as signal reporters could be designed for the detection of a target analyte or a biological process that is able to directly or indirectly trigger gold nanoparticle aggregation.

Trypsin is a pancreatic serine protease with a molecular weight of 24 kilo Daltons (kDa). Detection of trypsin has clinical significance since augmented levels of immunoreactive trypsin in the bloodstream can indicate pancreatic malfunction and this indicator is used to screen for and diagnose Cystic Fibrosis in newborns. Using the detection of proteases such as trypsin, thrombin and protease K as examples, we disclose in the present invention a colloidal gold nanoparticle-based colorimetric assay that provides a simple, sensitive, specific, and inexpensive approach for both visual and quantitative detection of proteases and protease inhibitors. Due to the biological significance of serine proteases we choose trypsin as one model for testing in the present invention; however, the results are applicable to all proteases and protease inhibitors. We have also tested aspects of the present invention using the proteases thrombin and protease K as discussed below. We believe the assay methods described can be used to detect activity of all known proteases and protease inhibitors. In addition, the inventive assay methods provide a means for detection of new proteases in samples and for detection of new protease inhibitors. The detection of new proteases and new protease inhibitors using the present methods would not necessarily be quantitative initially, but the qualitative nature of the assay methods would allow one to determine if there was proteolytic activity on a test peptide substrate and inhibition of the activity of a known or unknown protease using the methods described herein. Subsequent assays according to the present invention can be used to make quantitative measurements. We also believe that the present methods will be useful in testing for the active site specificity of proteases. For example, using a variety of peptide substrates with different sequences one can investigate the amino acid sequence requirements and specificity of a protease of interest, potential inhibitory amino acids, like a carboxyl terminal proline is for the activity of trypsin, can also be detected. Knowing the sequence requirements can help in elucidating the active site sequence of a protease. The present methods by providing a rapid visual signal and by allowing for quantitative data will be a valuable tool for investigating both proteolytic activity and inhibition of proteolytic activity and also in the development of assays for a wide variety of proteases.

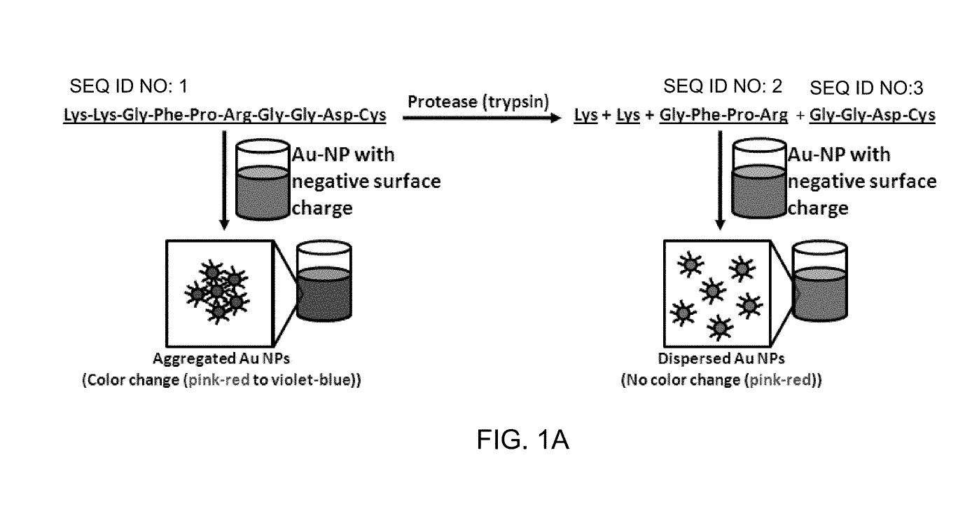

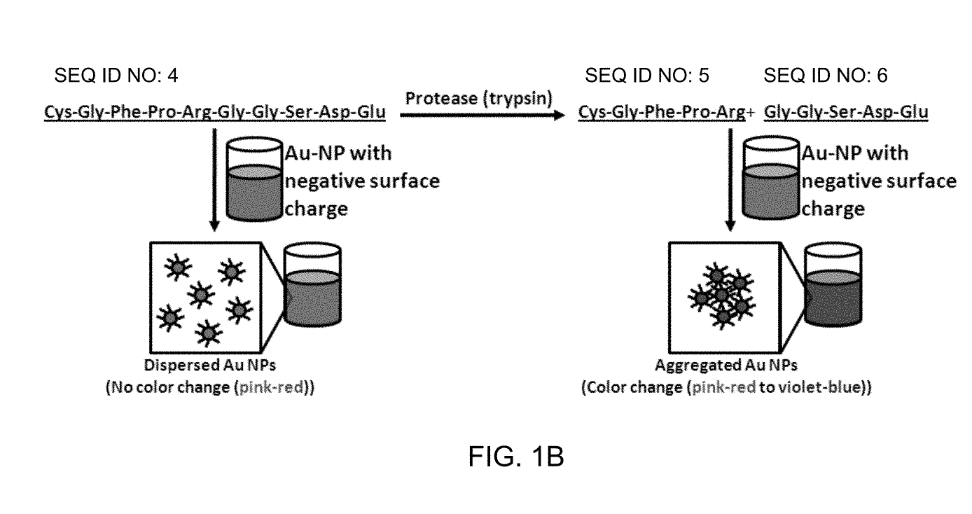

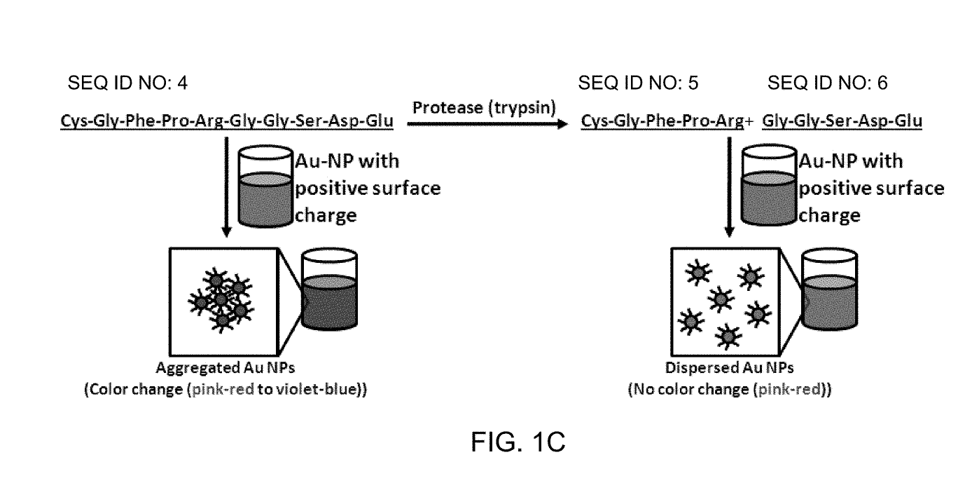

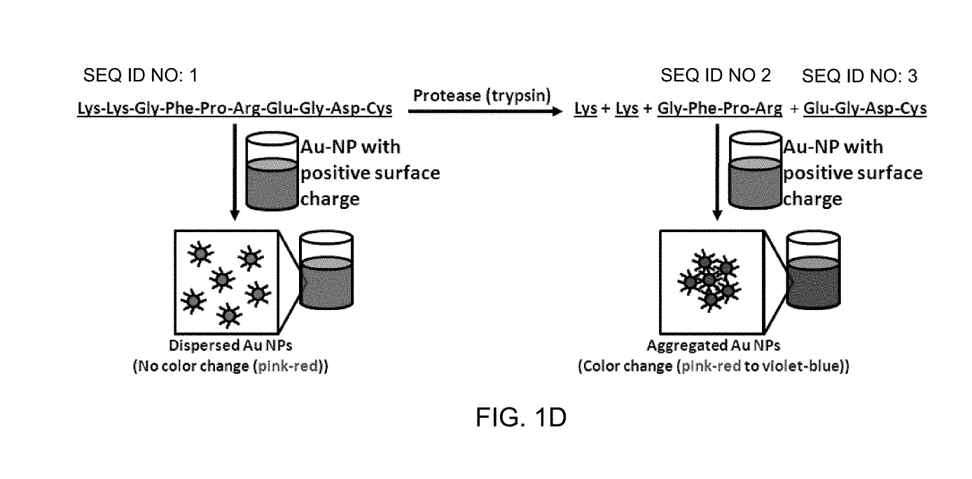

As shown in FIGS. 1a through 1d, the mechanism of signal generation in the present assay relies on hydrolytic cleavage of a peptide substrate by a protease of interest. The assay requires that either the peptide substrate or one or more of its hydrolytic fragments be able to cause aggregation of the gold nanoparticles. The initial colloidal gold nanoparticles can either have a positive or negative surface charge. The charge can vary from one preparation to another, for example, some samples of negatively charged nanoparticles prepared according to the present invention had a charge of −40 millivolts. The peptide substrate must include: at least a first functional group capable of covalently bonding to gold nanoparticles; a recognition sequence, i.e. cleavage site for the protease of interest; and the ability of either the intact peptide substrate or at least one of its hydrolytic fragments to aggregate the gold nanoparticles through a crosslinking mechanism. Peptide substrates can covalently bond to gold nanoparticles through thiol bonds from cysteine or methionine, via amine bonds between any number of amino acids and the gold nanoparticles, or through a modified amino acid that includes phosphine or disulfide linkages to gold nanoparticles. As discussed above, the functional group in the peptide substrate that covalently bonds to the nanoparticles can either be on one of the ends or it can be exposed on an end of one of the cleavage fragments. The peptide sequences also need to include recognition sites or cleavage sites for the protease of interest. As discussed above, the amino acid identity of a recognition sequence can be used to elucidate structure-activity data about the protease of interest. Some proteases have the ability to cleave based on a single amino acid while others require a sequence of two or more amino acids. The amino acid sequence in the present assays aids in creating the specificity of the assay so that one knows the protease of interest is the one being measured. Finally, either the initial peptide sequence or at least one of its hydrolytic fragments needs to be able to induce aggregation of the gold nanoparticles. The aggregation is believed to occur through crosslinking of gold nanoparticles wherein one amino acid of the aggregating species binds covalently to a first gold nanoparticle and a second portion of the aggregating species forms an ionic bond with the surface charges of another gold nanoparticle thereby crosslinking the two particles. The second portion comprises one or more amino acids having a net charge that is opposite to that of the surface charge of the nanoparticles.

In the present specification and claims either the accepted three letter abbreviations for each amino acid or their single letter abbreviations will be used and any sequences are written via the convention that the left end of the sequence is the amino terminal end while the right end is the carboxyl terminal end. Although the exact nature of the induced aggregation is not known, as stated above, it is theorized to involve both covalent bonding of the aggregation-inducing species to the surface of a gold nanoparticle and electrical charge interactions between the surface charges of the nanoparticles and opposite charges on the aggregation-inducing species that leads to formation of an ionic bond between the nanoparticles and the particular amino acid sequence. So for example, as discussed below, the peptide substrate SEQ ID NO: 1 Lys-Lys-Gly-Phe-Pro-Arg-Gly-Gly-Asp-Cys induces the aggregation of gold nanoparticles having a negative surface charge. After exposure of the peptide substrate to the protease trypsin the induced aggregation is reduced demonstrating that the hydrolytic fragments are not able to crosslink the nanoparticles. The peptide substrate SEQ ID NO: 1 Lys-Lys-Gly-Phe-Pro-Arg-Gly-Gly-Asp-Cys will be cleaved by the protease trypsin into the following fragments after complete digestion: SEQ ID NO: 2 Lys, Lys, SEQ ID NO: 3 Gly-Phe-Pro-Arg, and Gly-Gly-Asp-Cys. The Cys will bind to the surface of gold nanoparticles with very high affinity via covalent thiol bonding. It is expected that each gold nanoparticle can accommodate a plurality of peptide substrates bound to it through the Cys via a thiol bond. At a neutral, physiological pH of 7.4 the amino acids Asp and Glu are expected to have a net negative charge on their side chains. The amino acids Arg, Lys, and His are expected to have a net positive charge on their side chains at a pH of 7.4; however, given its pKa His is expected to carry a positive charge on only about 10% of the available side chains with 90% being neutral. So for practical purposes the majority of the His side chains in a sequence will be electrically neutral at a neutral pH. Thus, at a neutral pH the peptide substrate Lys-Lys-Gly-Phe-Pro-Arg-Gly-Gly-Asp-Cys is expected to bond to gold nanoparticles via a thiol bond from the Cys and will have positive charges from 2 Lys and 1 Arg for a total of three positive charges wherein two are at the amino terminal end of the peptide substrate. The Asp is expected to provide a negative charge to the peptide substrate. The second bond is theorized to occur as an ionic bond between one or more amino acids and the surface charges of the nanoparticle. Therefore, in the present example the second bond(s) will be ionic most likely through the two terminal end Lys, which each carry a positive charge, and the negative surface charge of the gold nanoparticles. Although there might be some interaction with the internal Arg, it is theorized that this is minimal compared to the terminal Lys. As trypsin cleaves the peptide substrate the fragments have greatly reduced or no ability to form an ionic bond to the nanoparticles. The loss of a single Lys still leaves one Lys to bond, but after loss of the second Lys the internal Arg is unlikely to form much of a bond so crosslinking and aggregation is lost. The final peptide substrate bound to the gold nanoparticle, assuming complete cleavage, is SEQ ID NO: 3 Gly-Gly-Asp-Cys. Since the Asp will be carrying net negative charge this means nanoparticles with bound peptide fragment will be even more repelled from each other than the original nanoparticles, which carry a net negative surface charge. Following in the current theory one can envision creating a peptide substrate having on one of its terminal ends one or more of the amino acids Glu or Asp and on the other end a Cys. This peptide substrate should be able to induce aggregation of gold nanoparticles that have a positive surface charge through a similar mechanism to that discussed above. The only difference is that now the peptide substrate has a negative charge and can form an ionic bond with a positively charged nanoparticle. Again, if one designs the peptide substrate with appropriately placed Lys and Arg one can cause cleavage of the Asp and Glu by trypsin and loss of the ability of the peptide substrate to cross link and aggregate the gold nanoparticles.

Following in the same theoretical vein as above, one can envision creating a peptide substrate wherein the sequence might be, for example, SEQ ID NO: 4 Cys-Gly-Phe-Pro-Arg-Gly-Gly-Ser-Asp-Glu. This would be expected to covalently bond to gold nanoparticles having a negative surface charge through the amino acid Cys; however, the terminal Asp and Glu would prevent the intact peptide from forming ionic bonds to other gold nanoparticles having a negative surface charge. Therefore the intact peptide should not be able to cause aggregation of the gold nanoparticles. Following exposure to trypsin and after complete cleavage the resulting proteolytic fragments should comprise: SEQ ID NO: 5 Cys-Gly-Phe-Pro-Arg and SEQ ID NO: 6 Gly-Gluy-Ser-Asp-Glu. It is expected that the first fragment will now cause aggregation of the gold nanoparticles. One bond will be a covalent thiol linkage between the Cys and the nanoparticle and the other will be ionic between the terminal Arg carrying a positive charge and the negative surface charge of the gold nanoparticles. Now one can also envision a peptide substrate with a sequence to allow the same aggregation principals to apply except one uses gold nanoparticles having a positive surface charge and the intact peptide has one or more Arg on a terminal end and the action of the enzyme removes the Arg exposing negatively charged amino acids such as Asp or Glu.

To further illustrate the invention as described above, several of the concepts are shown schematically in FIGS. 1a, 1b, 1c, and 1d. As shown in FIGS. 1a and 1c, in one embodiment of the present invention, the intact peptide substrate causes aggregation of the gold nanoparticles having either a negative surface charge or a positive surface charge, respectively. In the non-aggregated state the dispersed gold nanoparticle solution has a pink-red color, for nanoparticles having an average diameter of 20 nm the absorbance band peak is at about 520 nm±5 nm. Upon aggregation the color of the gold nanoparticle solution will change to a violet-blue color detectable with the naked eye. This color change is also seen as a shift in the absorbance band maximum to higher wavelengths in the range of from 600 to 750 nm. Using a series of known concentrations of peptide substrate with a constant level of gold nanoparticles one can create a standard curve to quantify the amount of peptide substrate in a test solution. Also as shown in the figures, the presence and amount of the protease of interest will be detected by its ability to cause a reduction in the aggregation caused by a standard level of the peptide substrate after pre-exposure of the peptide substrate to the protease solution prior to addition to the nanoparticles since the hydrolytic fragments cannot induce aggregation of the gold nanoparticles. As shown, the assay procedure can be used on both positive and negative surface charged gold nanoparticles. One will have to alter the peptide sequence to provide for the proper amino acids to permit ionic bonding as described above depending on whether the gold nanoparticles have a positive or negative surface charge as shown in FIGS. 1c and 1a respectively. Understanding the assay procedure described above, it is also clear how one might use the assay to detect the presence of a protease inhibitor using known amounts of peptide substrate and known amounts of the protease of interest. The presence of the inhibitor will reduce the amount of hydrolysis and therefore alter the degree of aggregation compared to standard samples prepared using known amounts of peptide substrate and protease. The flexibility of the present inventive assay is wide. One can vary the amount of peptide substrate, the reaction or exposure times, the reaction and exposure temperature and other reaction parameters to shift the sensitivity of the assay depending on the levels of protease or inhibitor that are being measured. Generally, for mammalian enzymes the reaction temperatures for hydrolytic cleavage reactions and for interaction of protease inhibitors with the protease the temperatures are from 10 to 65° C. for times of from 10 minutes to 3 hours. For cold tolerant proteases the reaction temperatures can be as low as 0° C., while heat tolerant proteases can be measured at 95 to 100° C. over the same time periods.

In FIGS. 1b and 1d an alternative embodiment is shown wherein the intact peptide sequences do not cause aggregation of the gold nanoparticles, instead the hydrolytic fragments cause the aggregation. In this embodiment one also measures the shift in absorbance band maximum as an indicator of and for quantification of protease activity except it is moving in the opposite direction from the previous embodiment where the peptide substrate caused the aggregation. Again, one can utilize this assay when the gold nanoparticles have a positive or a negative surface charge. The assay is equally useful to detect the presence and amount of a protease inhibitor. In this embodiment the ionic bonding portion of the peptide substrate is not on or near one of terminal ends in the intact peptide substrate so there are no crosslinking bonds being formed. In fact for the examples shown, the intact peptide acts to enhance the repulsion between nanoparticles since it carries on one end the same charges as the surface charges of the nanoparticles. Upon cleavage one of the fragments includes both a Cys to permit covalent bonding to a gold nanoparticle and either positively charged amino acids or negatively charged amino acids depending on the surface charge of the nanoparticles. As discussed above the Cys could also be located internally in the intact peptide substrate and exposed only via action of the protease. In this case again you would have a situation wherein the intact peptide substrate has only one or none of the groups capable of forming either a covalent or ionic bond with the gold nanoparticles exposed. Then one or more of the hydrolytic fragments has both the covalent bonding group and the ionic bonding group available for bonding so the crosslinking reaction can occur and the nanoparticles are aggregated by one or more of the hydrolytic fragments.

As discussed above, the overwhelming majority of commercially available gold nanocolloids are prepared by the standard sodium citrate reduction reaction. This method is a bottom-up method and allows for the synthesis of spherical gold nanoparticles with diameters ranging from 1 to 200 nanometers (nm) which are capped with negatively charged citrate ions. The capping controls the growth of the nanoparticles in terms of rate, final size, geometric shape and electrostatic repulsion stabilizes the nanoparticles against aggregation.

In contrast to the prior process of bottom-up fabrication using wet chemical processes, gold nanocolloids used in the present invention are preferably produced by a top-down nanofabrication approach. The top-down fabrication methods of the present invention start with a bulk material in a liquid and then break the bulk material into nanoparticles in the liquid by applying physical energy to the material. The physical energy can be mechanical energy, heat energy, electric field arc discharge energy, magnetic field energy, ion beam energy, electron beam energy, or laser beam energy including laser ablation of the bulk material. The top-down process produces a pure, bare colloidal gold nanoparticle that is stable in the ablation liquid and avoids the wet chemical issues of residual chemical precursors, stabilizing agents and reducing agents. These particles tend to carry a negative surface charge naturally. They can be converted to a positive surface charges through use of surfactants to coat the nanoparticles as known to those of ordinary skill in the art.

The top-down nanofabrication approaches all require that the generation of the nanoparticles from the bulk material occur in the presence of a suspension medium. In one embodiment, the process comprises a one step process wherein the application of the physical energy source, such as mechanical energy, heat energy, electric field arc discharge energy, magnetic field energy, ion beam energy, electron beam energy, or laser energy to the bulk gold occurs in the suspension medium. The bulk source is placed in the suspension medium and the physical energy is applied thus generating nanoparticles that are immediately suspended in the suspension medium as they are formed. In another embodiment, the present invention is a two-step process including the steps of: 1) fabricating gold nanoparticle arrays on a substrate by using photo, electron beam, focused ion beam, nanoimprint, or nanosphere lithography as known in the art; and 2) removing the gold nanoparticle arrays from the substrate into the suspension liquid using one of the above described physical energy methods. Tabor, C., Qian, W., and El-Sayed, M. A., Journal of Physical Chemistry C, Vol 111 (2007), 8934-8941; Haes, A. J.; Zhao, J.; Zou, S. L.; Own, C. S.; Marks, L. D.; Schatz, G. C.; Van Duyne, R. P. Journal Of Physical Chemistry B, Vol 109 (2005), 11158. In both the one step and two step methods the colloidal gold is formed in situ by generating the nanoparticles in the suspension medium using one of the physical energy methods.

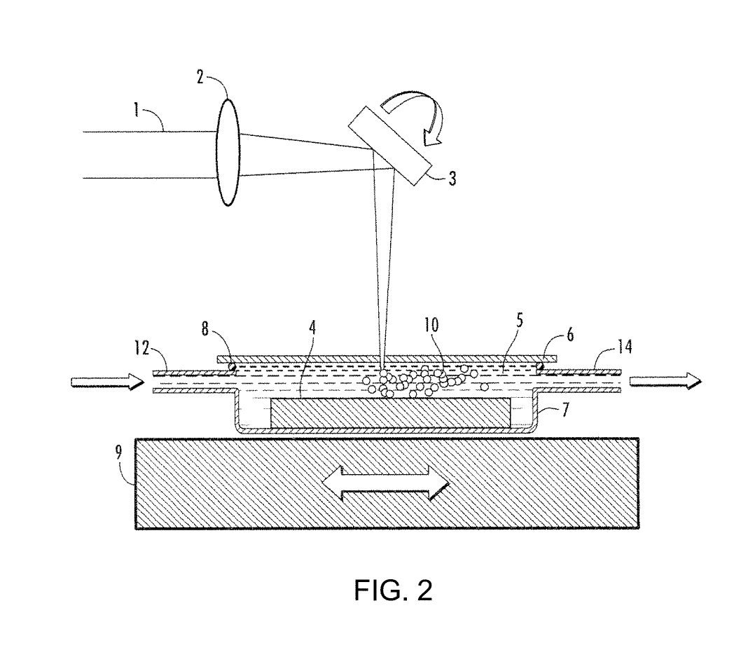

In at least one embodiment of the present invention, gold nanocolloids were produced by pulsed laser ablation of a bulk gold target in deionized water as the suspension medium. FIG. 2 schematically illustrates a laser-based system for producing colloidal suspensions of nanoparticles of complex compounds such as gold in a liquid using pulsed laser ablation in accordance with the present invention. In one embodiment a laser beam 1 is generated from an ultrafast pulsed laser source, not shown, and focused by a lens 2. The source of the laser beam 1 can be a pulsed laser or any other laser source providing suitable pulse duration, repetition rate, and/or power level as discussed below. The focused laser beam 1 then passes from the lens 2 to a guide mechanism 3 for directing the laser beam 1 at a target 4 of the bulk material. Alternatively, the lens 2 can be placed between the guide mechanism 3 and a target 4 of the bulk material. The guide mechanism 3 can be any of those known in the art including piezo-mirrors, acousto-optic deflectors, rotating polygons, a vibration mirror, or prisms. Preferably the guide mechanism 3 is a vibration mirror 3 to enable controlled and rapid movement of the laser beam 1. The guide mechanism 3 directs the laser beam 1 to a target 4. In one embodiment, the target 4 is a bulk gold target. The target 4 is submerged a distance, from several millimeters to preferably less than 1 centimeter, below the surface of a suspension liquid 5. The target 4 is positioned in a container 7 additionally but not necessarily having a removable glass window 6 on its top. Optionally, an O-ring type seal 8 is placed between the glass window 6 and the top of the container 7 to prevent the liquid 5 from leaking out of the container 7. Additionally but not necessarily, the container 7 includes an inlet 12 and an outlet 14 so the liquid 5 can be passed over the target 4 and thus be re-circulated. The container 7 is optionally placed on a motion stage 9 that can produce translational motion of the container 7 with the target 4 and the liquid 5 relative to the laser beam 1. Flow of the liquid 5 is used to carry the nanoparticles 10 generated from the target 4 out of the container 7 to be collected as a colloidal suspension. The flow of liquid 5 over the target 4 also cools the laser focal volume. The liquid 5 can be any liquid that is largely transparent to the wavelength of the laser beam 1, and that serves as a colloidal suspension medium for the target material 4. In one embodiment, the liquid 5 is deionized water having a resistivity of greater than 0.05 MOhm·cm, and preferably greater than 1 MOhm·cm. In other embodiments the liquid 5 can comprise other suspension liquids including, for example, a physiological buffer solution, a phosphate buffered saline or other suitable media. The system thus allows for generation of colloidal gold nanoparticles in situ in a suspension liquid so that a colloidal gold nanoparticle suspension is formed. The formed gold nanoparticles are immediately and stably suspended in the liquid and thus no dispersants, stabilizer agents, surfactants or other materials are required to maintain the colloidal suspension in a stable state. This result allows the creation of a unique colloidal gold suspension that contains bare gold nanoparticles. Generally, gold nanoparticles formed by this method have a negative surface charge. As known to those of skill in the art, if one wants positively surface charged nanoparticles from the normally negatively charged ones, one can alter the surface charge using surfactant coatings.

The following laser parameters were used to fabricate gold (Au) nanocolloids by pulsed laser ablation of a bulk gold target in deionized water: a pulse energy of 10 micro Joules (μJ), a pulse repetition rate of 100 kHz, a pulse duration of 700 femtoseconds (fs), and a laser spot size on the ablation target of about 50 microns (μ). For the preparation of Au nanocolloids according to the present invention, a 16 millimeter (mm) long, 8 mm wide, and 0.5 mm thick rectangular target of Au from Alfa Aesar was used. For convenience, the Au target materials can be attached to a bigger piece of a bulk material such as a glass slide, another metal substrate, or a Si substrate.

More generally, for the fabrication of gold nanocolloids used in the present invention, the suitable laser ablation parameters are as follows: a pulse duration in a range of from about 10 fs to about 500 picoseconds (ps), preferably from about 100 fs to about 30 ps; a pulse energy in the range of from about 1 μJ to about 100 μJ; a pulse repetition rate in the range of from about 10 kHz to about 10 MHz; and the laser spot size may be less than about 100 μm. The target material has a size in at least one dimension that is greater than a spot size of a laser spot at a surface of the target material.

Samples of colloidal gold nanoparticles prepared by laser ablation in deionized water were characterized by an array of commercially available analytic instruments and techniques, including UV-VIS absorption spectra, dynamic light scattering (DLS), and transmission electron microscopy (TEM). UV-VIS absorption spectra were recorded with a Shimadzu UV-3600 UV-VIS-NIR spectrophotometer. DLS measurements were performed using a Nano-ZS90 Zatasizer (Malvern Instrument, Westborough, Mass.). Gold nanoparticles were visualized using transmission electron microscopy (TEM; JEOL 2010F, Japan) at an accelerating voltage of 100 kilovolts (kV). All measurements and processes were carried out at room temperature, approximately 20° C.

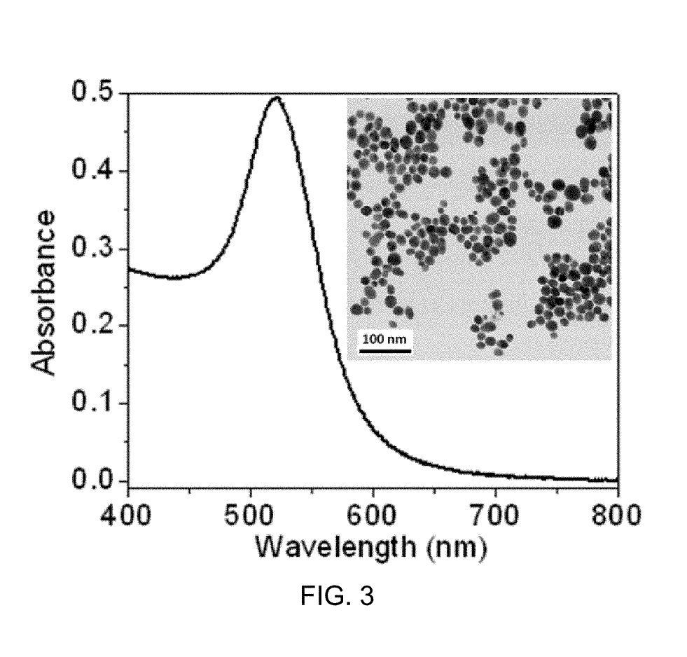

FIG. 3 shows the UV-VIS absorption spectrum and a Transmission Electron Microscopy (TEM) picture of a stable bare colloidal gold nanoparticle preparation prepared by laser ablation in deionized water according to the present invention. The maximum of localized surface plasmon resonance of the prepared colloidal gold nanoparticle is at 520 nm±5 nm. The average Feret diameter of the nanoparticles was determined to be 20.8 nm as measured from TEM images like the one shown in the inset. This solution has a visible pink-red color. In the present specification all of the experiments involving gold nanocolloids were conducted using gold nanocolloids at a concentration of 1 nanomolar (nM).

Since trypsin, a pancreatic serine protease with molecular weight of 24 kDa, has great clinical significance in terms of the diagnosis of pancreatic malfunction and as a screening marker for the diagnosis of Cystic Fibrosis, the detection of trypsin activity was used as one of the model systems for demonstrating the embodiments of the present invention. In addition, the present invention was demonstrated using the proteases thrombin and protease K as described below. Other proteases are believed to work equally well for the development and application of the inventive colloidal gold nanoparticle-based colorimetric assay for the detection of their proteolytic activity. The discussed detection strategies for use of the present inventive colloidal gold nanoparticle-based assay are however of a general nature and apply in the same way to the detection of other proteases such as those comprising, but not limited to, chymotrypsin, thrombin, prostate-specific antigen, HIV-1 protease, elastase, metalloendopeptidases, and subtilisin.

A first step in the present invention is the design and preparation of a peptide substrate specific for the protease of interest, for example trypsin. A suitable peptide substrate needs to meet several criteria. First, it needs to include at least one first functional group on or near its amino or carboxyl terminus that covalently binds to gold nanoparticles. As discussed above, thiol groups, amine functions, phosphine functions and disulfide functions all covalently bond to various degrees to gold nanoparticles. Thiol groups are considered to show the highest affinity for gold surfaces, approximately 200 kiloJoules/mole (kJ/mol), and therefore a majority of gold nanoparticle surface functionalization occurs through using ligand molecules having thiol groups which bond to the surfaces of gold nanoparticles via a thiol-Au bond. Therefore, in our design of the peptide substrate which could be selectively recognized and cleaved by trypsin, cysteine, a small thiol-containing amino acid, was included at the amine or carboxyl-terminus and the peptide substrates were bound onto the surface of the gold nanoparticles via the thiol group of the cysteine residue. As discussed above, in the embodiment wherein one or more hydrolytic fragments cause the aggregation the Cys could be located internally in the peptide substrate. Another thiol-containing amino acid that could have been used is methionine. Other functional groups could be used, however, cysteine has the advantage of being a high affinity binder to gold with a relatively low cost and thus it was chosen. The rest of the peptide substrate needs to accomplish two tasks, it needs to have the cleavage site recognized by the protease and either the intact peptide substrate or one or more of its hydrolytic fragments need to cause aggregation of the gold nanoparticles. As discussed above the aggregation is theorized to occur via an ionic bond in conjunction with the covalent bond in the present invention. Thus the surface charge on the gold nanoparticles, either positive or negative, can influence the design of the rest of the peptide substrate sequence by determining what charged amino acids need to be in the peptide substrate to form the ionic bond. The cleavage site for trypsin, the model protease used in the present experiments, is the carboxyl side of arginine and lysine residues that are not adjacent to a carboxyl-terminal proline. The non-aggregated gold nanoparticles having a size of 20 nm have a pink-red color and an absorbance band maximum near 520 nm±5 nm. As these nanoparticles aggregate the color shifts to a violet-blue and the absorbance band moves to a new maximum absorbance at a wavelength of about 650 nm, depending on the peptide substrate.

FIGS. 1a to 1d show the four main types of assays according to the present invention. In FIGS. 1a and 1c the peptide substrate causes aggregation of the gold nanoparticles, both with a negative and a positive nanoparticle surface charge. The peptide substrate needed to be different to accomplish this because of the surface charge differences. The peptide substrates have either positively charged amino acids or negatively charged amino acids on one end. As shown in FIGS. 1b and 1d an alternative embodiment relies on the peptide substrate not aggregating the gold nanoparticles, instead the hydrolytic fragments cause aggregation. In these embodiments the cleavage activity exposes the ionic bonding groups that are internal in the intact peptide. Again the exposed charges are of the opposite sign of the surface charge of the nanoparticles.

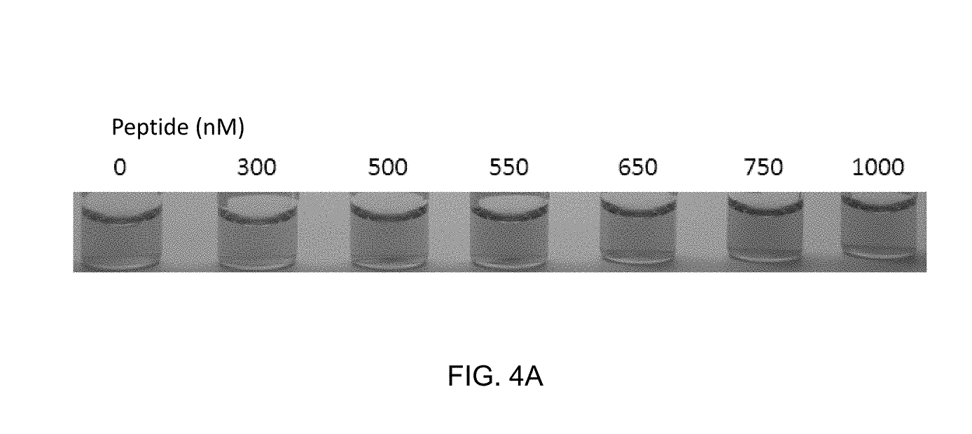

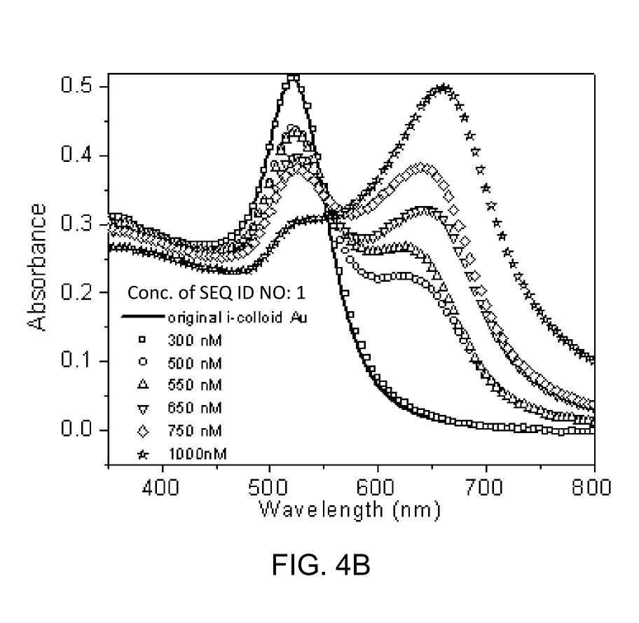

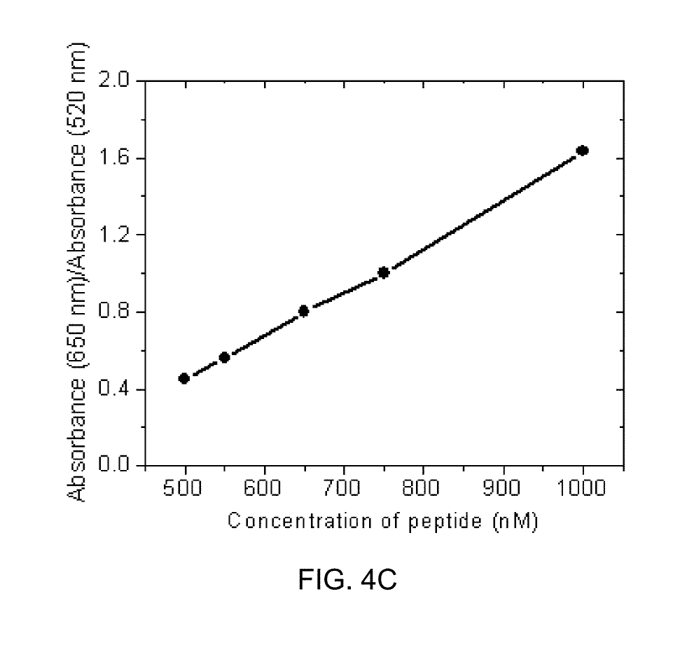

Based on the design criteria for the peptide substrate mentioned above, a sequence of amino acids in the peptide substrate of SEQ ID NO: 1 Lys-Lys-Gly-Phe-Pro-Arg-Gly-Gly-Asp-Cys was used in an assay method patterned after FIG. 1a. The effects of this peptide substrate on a solution of gold nanoparticles are shown in FIGS. 4a, 4b and 4c. Unless noted otherwise, all incubations and reactions reported in the present specification were conducted at 20° C. The final reaction volume in all experiments described in the present specification, unless noted otherwise, was 500 microliters and the gold nanoparticle concentration was 1 nM. The concentrations of the peptide substrate, proteases or inhibitors described in the figures and specification are the final concentrations in the 500 microliter reaction volume. The aggregation reaction is quite rapid and is completed in 30 minutes or less. Thus, in all of the described experiments the aggregation state of any gold nanoparticle solution could be measured after 30 minutes or less. As known to one of ordinary skill in the art enzymatic reactions generally increase with increasing temperature until the temperature approaches the denaturation temperature for the enzyme. For most mammalian enzymes the enzymes can function in a temperature range of from about 10 to 65° C. The present assays, however, can also be used to investigate both cold-tolerant and heat-tolerant proteolytic enzymes. For example, some thermostable bacteria have proteolytic enzymes that can function at temperatures of 95 to 100° C. and other bacteria can be found with enzymes that function at temperatures as low as 0° C. All of these can be evaluated using the present inventive assay methods. The experimental data shown in FIGS. 4a to 4c confirm that this peptide substrate did induce aggregation of gold nanoparticles with an average diameter of 20 nm after being added to solutions of colloidal gold nanoparticles in a linear, concentration dependent manner that was also very visible to the naked eye. FIG. 4a is a series of photographs demonstrating generation of colloidal gold nanoparticle solutions with different colors depending on the concentration of peptide substrate added to the gold nanoparticle solutions. The tonality of the solution of colloidal gold nanoparticles changes from pink-red on the left with 0 nM of peptide substrate and no aggregation to a violet-blue with an increasing concentration of the peptide substrate in the solution from 300 nM to 1000 nM and with increasing aggregation of the gold nanoparticles. FIG. 4b displays the corresponding ultraviolet-visible (UV-VIS) spectra of the solutions of colloidal gold nanoparticles in the presence of the peptide substrate at different concentrations ranging from 0 to 1000 nM. As the gold nanoparticles aggregate, the localized surface plasmon resonance of the Au nanoparticles with an average particle diameter of 20 nm is significantly modified with a decrease of the absorption band at 520 nm±5 nm and appearance of a new absorption band at a longer wavelength of around 650 nm due to dipole coupling between the localized surface plasmon resonance (LSPR) of neighboring particles forming the aggregates, which increases as the concentration of the peptide substrate in the solution of colloidal gold nanoparticles is increased from 300 nM to 1000 nM. The results displayed in FIG. 4b are summarized in FIG. 4c, which displays the linear dependence of the ratio of the absorbance at 650 nm to the absorbance at 520 nm of the solution of colloidal gold nanoparticles versus the concentration of the peptide substrate added to solution of colloidal gold nanoparticles in a range from 500 nM to 1000 nM. The linearity of this range shows that this absorbance ratio, 650 nm/520 nm, could be used for quantitative analysis of the amount of the protease trypsin by comparing the ratios of the absorbance at 650 nm to the absorbance at 520 nm of the ultraviolet-visible spectra of standard samples containing known amounts of the protease analyte to those of the experimental results from a sample with an unknown amount of trypsin.

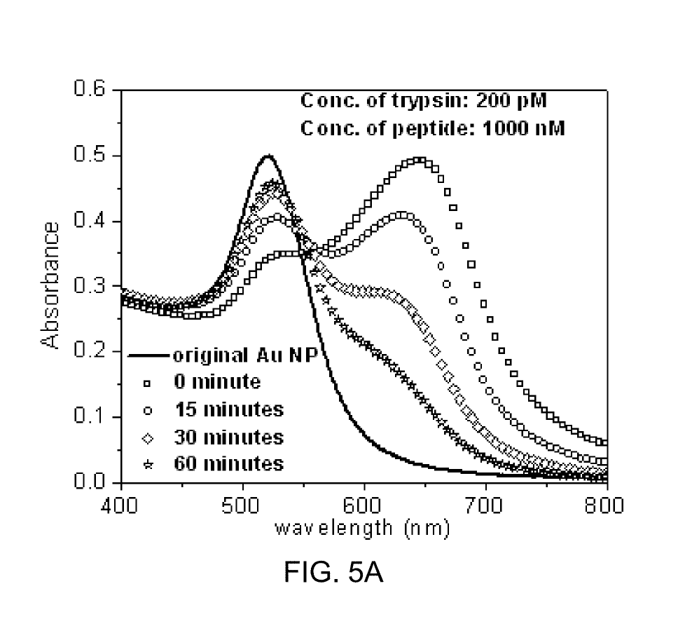

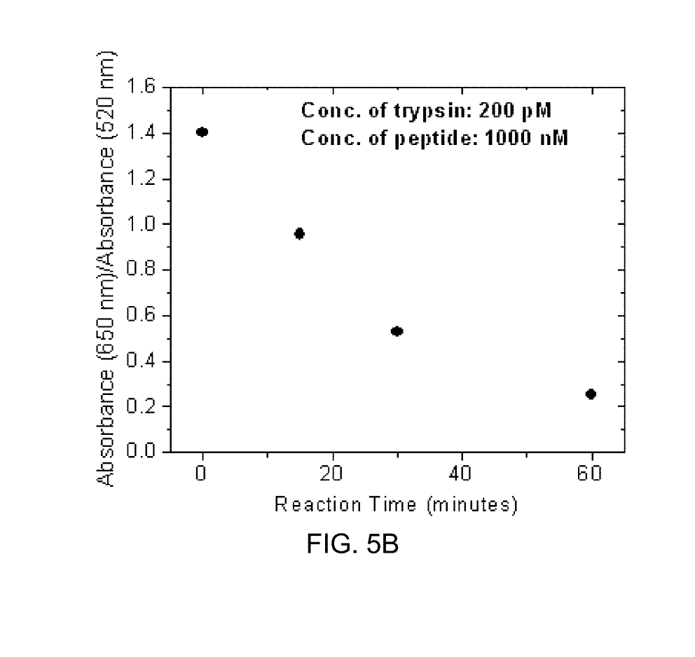

After confirming that the peptide substrate SEQ ID NO: 1 Lys-Lys-Gly-Phe-Pro-Arg-Gly-Gly-Asp-Cys did induce the aggregation of negative surface charged gold nanoparticles, we carried out another experiment of examining the ultraviolet-visible spectra of solutions of colloidal gold nanoparticles after they were added to solutions of peptide substrates that had been pre-exposed for different time periods of from 0 minutes (min) to 60 min to trypsin. The purposes of this experiment were to confirm that the proteolyzed peptide fragments after hydrolytic cleavage of peptide substrate SEQ ID NO: 1 Lys-Lys-Gly-Phe-Pro-Arg-Gly-Gly-Asp-Cys by trypsin could not cross-link negatively charged gold nanoparticles and to determine the kinetics of the hydrolytic cleavage of the peptide substrate SEQ ID NO: 1 Lys-Lys-Gly-Phe-Pro-Arg-Gly-Gly-Asp-Cys by trypsin. Since concentrations of both the peptide substrate and the trypsin control the kinetics of reaction of the hydrolytic cleavage, the concentrations of peptide substrate and trypsin used in this experiment were fixed at 1000 nM and 200 picomolar (pM), respectively, and the temperature was kept at 20° C. The results of this experiment are shown in the FIGS. 5a and 5b. FIG. 5a displays the ultraviolet-visible spectra of the solutions of the colloidal gold nanoparticles after being added to the solutions of the peptide substrate SEQ ID NO: 1 Lys-Lys-Gly-Phe-Pro-Arg-Gly-Gly-Asp-Cys pre-exposed to trypsin as described above for 0 min (□), 15 min (◯), 30 min (⋄), and 60 min (⋆). It is clearly shown in FIG. 5a that the longer the time period which peptide substrates were pre-exposed to trypsin, the lower the aggregation of the gold nanoparticles induced by addition of the peptide substrate, which proves that the proteolyzed peptide fragments of peptide substrate SEQ ID NO: 1 Lys-Lys-Gly-Phe-Pro-Arg-Gly-Gly-Asp-Cys after hydrolytic cleavage by trypsin do not cross-link negatively charged gold nanoparticles. The absorbance spectrum shifts back to a maximum at 520 nm±5 nm, non-aggregated nanoparticles, as the pre-exposure time increases. The expected fragments are shown in FIG. 1a and include SEQ ID NO: 2 Lys, SEQ ID NO: 3 Gly-Phe-Pro-Arg, and Gly-Gly-Asp-Cys. The results in FIG. 5a show a shift in the absorbance maximum from 650 nm to 520 nm±5 nm as the amount of peptide substrate is reduced by longer pre-exposures to trypsin. The results displayed in FIG. 5a are summarized in FIG. 5b, which displays the ratio of the absorbance at 650 nm to the absorbance at 520 nm of the solution of colloidal gold nanoparticles as a function of the time of pre-exposure of the peptide substrate to trypsin. It is shown that it took more than 60 min for complete hydrolytic cleavage of a solution of 1000 nM of the peptide substrate SEQ ID NO: 1 Lys-Lys-Gly-Phe-Pro-Arg-Gly-Gly-Asp-Cys by trypsin at a concentration of 200 pM.

The results discussed above confirm that the peptide substrate SEQ ID NO: 1 Lys-Lys-Gly-Phe-Pro-Arg-Gly-Gly-Asp-Cys could induce aggregation of negatively charged gold nanoparticles; that proteolyzed peptide fragments of the peptide substrate after hydrolytic cleavage of it by trypsin could not cross-link negatively charged gold nanoparticles; and show the kinetics of the hydrolytic cleavage of peptide substrate by trypsin.

Next we were interested in the limits of detection of this colloidal gold nanoparticle-based colorimetric assay for the detection of trypsin. The method of detecting a protease using this assay comprises a series of steps. First, a fluid, which may be a biological fluid, containing or suspected to contain trypsin is mixed with an aqueous solution of the peptide substrate to generate a mixture and this is incubated at room temperature, which is about 20° Celsius, for a sufficient amount of time, for example between 0.5 to 3 hours, to enable hydrolytic cleavage of a portion of the peptide substrate by the trypsin. It is important for the determination to be accurate that one not run out of substrate or move into a non-linear portion of the hydrolytic reaction kinetics. In a second step after incubation for the selected time a colloidal suspension of gold nanoparticles with a pink-red color, meaning non-aggregated, is added to the mixture and depending on the amount of peptide substrate remaining uncleaved, which depends on the amount of trypsin in the fluid, a particular state of aggregation of the gold nanoparticles is induced, resulting in the formation of a final solution with a characteristic color in 30 minutes or less. The third step is qualitatively detecting the amount of trypsin in the biological fluid via comparing the characteristic color of the final solution to the colors of standard samples containing known amounts of trypsin and peptide substrate exposed to the same conditions. The last step is quantitatively detecting the amount of trypsin in the fluid by examining the ultraviolet-visible spectrum of the final solution and comparing the ratio of the absorbance at 650 nm to the absorbance at 520 nm of the final solution to ratios of the absorbance at 650 nm to the absorbance at 520 nm of standard samples containing known amounts of trypsin and peptide substrate exposed to the same conditions.

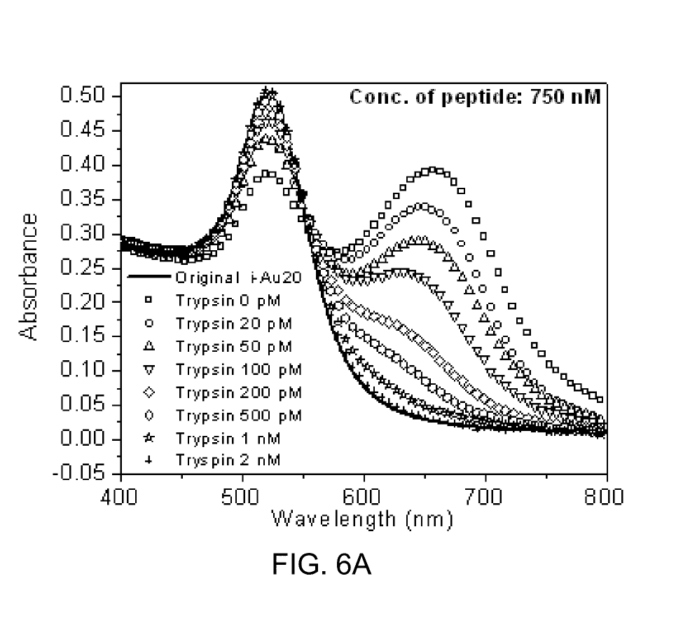

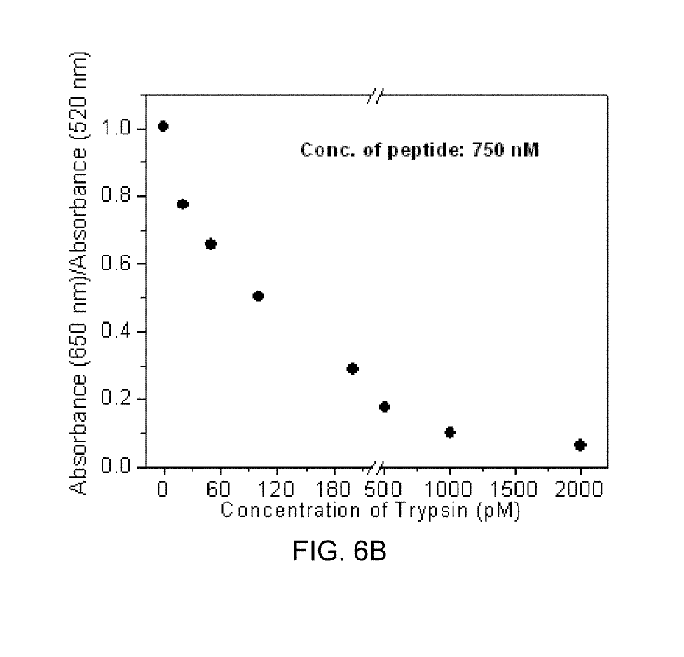

The experimental results shown in FIGS. 6a and 6b were directed toward determining the sensitivity and limits of detection for the assay method. In this experiment, the level of peptide substrate was kept constant at 750 nM, the reaction time was set at 60 minutes and the reaction temperature was 20° C. The amount of trypsin was varied from 20 picomolar (pM) to 2 nM. FIG. 6a displays the ultraviolet-visible spectra of the solutions of colloidal gold nanoparticles after being added to solutions of the peptide substrate SEQ ID NO: 1 Lys-Lys-Gly-Phe-Pro-Arg-Gly-Gly-Asp-Cys which were pre-exposed to trypsin. The gold nanoparticles used had a negative surface charge and an average diameter of 20 nm. The results displayed in FIG. 6a are summarized in FIG. 6b, which displays the ratio of the absorbance at 650 nm to the absorbance at 520 nm of the solutions of colloidal gold nanoparticles after being added to the solutions of the peptide substrate SEQ ID NO: 1 Lys-Lys-Gly-Phe-Pro-Arg-Gly-Gly-Asp-Cys pre-exposed to trypsin for 60 min as a function of the concentration of trypsin in the pre-exposure. The data shown in FIGS. 6a and 6b suggest that for these reaction conditions, the limit of detection of the colloidal gold nanoparticle-based colorimetric assay developed in the present invention for sensing trypsin is about 20 pM of trypsin. With no trypsin present and a concentration of 750 nM of the peptide substrate the ratio of the absorbance at 650 nm/absorbance at 520 nm was essentially 1 meaning that the two absorbances were equal. As the amount of trypsin is increased the absorbance band shifts toward what the gold nanoparticles show in the absence of any peptide and the ratio approaches a value of about 0.05.

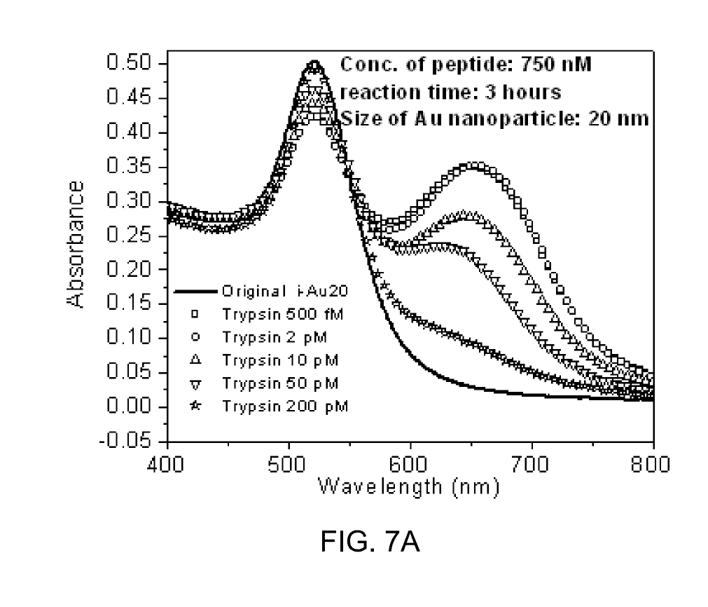

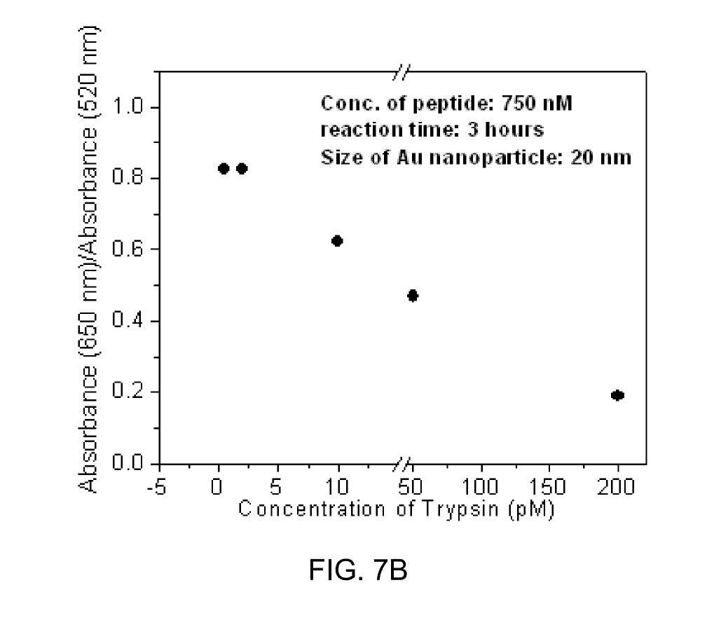

In the experiment described above, the peptide substrate SEQ ID NO: 1 Lys-Lys-Gly-Phe-Pro-Arg-Gly-Gly-Asp-Cys was exposed to the trypsin for 60 minutes. In an attempt to extend the range of the assay sensitivity, we carried out another experiment wherein the time of pre-exposure to the trypsin was increased from 1 hour to 3 hours using the same levels of peptide substrate, gold nanoparticles and the same temperature. The experimental data shown in the FIGS. 7a and 7b illustrate that limit of the detection of trypsin using the gold nanoparticle-based colorimetric assay could be improved by increasing the time period over which the peptide substrate SEQ ID NO: 1 Lys-Lys-Gly-Phe-Pro-Arg-Gly-Gly-Asp-Cys was exposed to trypsin. In these experiments the levels of trypsin were varied in the range of from 500 femtomolar (fM) to 200 pM. FIG. 7a displays the ultraviolet-visible spectra of the solutions of colloidal gold nanoparticles after being added to solutions of the peptide substrate SEQ ID NO: 1 Lys-Lys-Gly-Phe-Pro-Arg-Gly-Gly-Asp-Cys pre-exposed to trypsin at different concentrations of from 500 fM to 200 pM for 3 hours. The gold nanoparticles used had an average diameter of 20 nm. The results displayed in FIG. 7a are summarized in FIG. 7b, which displays the ratio of the absorbance at 650 nm to the absorbance at 520 nm of the solutions of colloidal gold nanoparticles after being added to the solutions of peptide substrate SEQ ID NO: 1 Lys-Lys-Gly-Phe-Pro-Arg-Gly-Gly-Asp-Cys pre-exposed to trypsin for 3 hours as a function of the concentration of trypsin. The data demonstrates that increasing the pre-exposure reaction time period from 60 minutes to 3 hours improved the limit of detection of trypsin using the colloidal gold nanoparticle-based colorimetric assay developed in the present invention from 20 pM to approximately 2 to 10 pM.

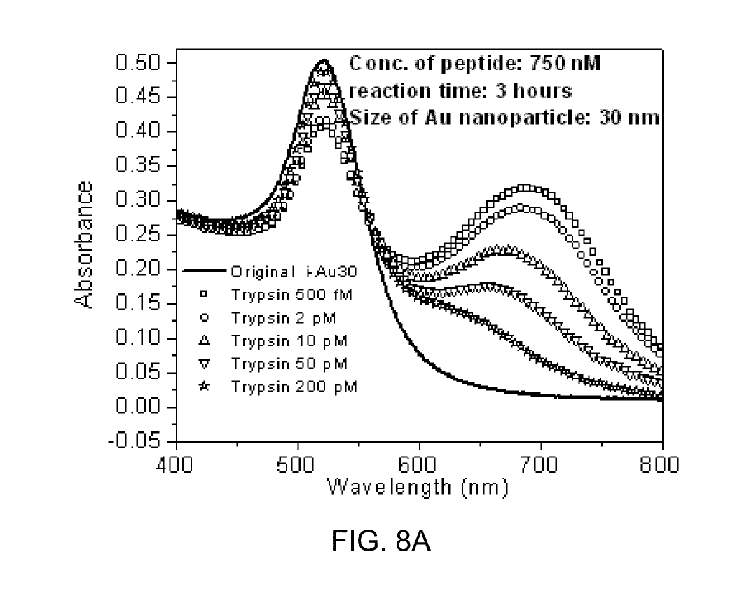

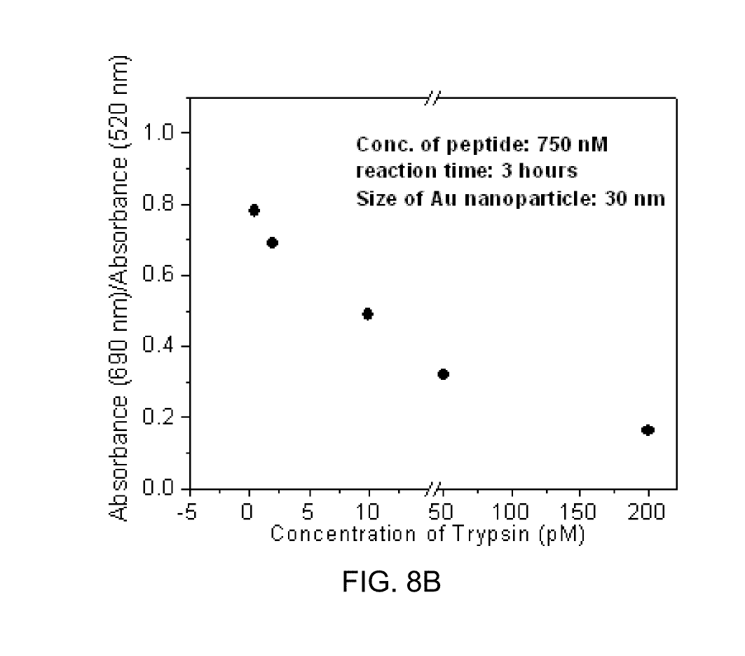

In the experiments described and shown in FIGS. 3-7 the gold nanoparticles had an average diameter of 20 nm. In another experiment the effect of nanoparticle size on the limits of detection was investigated. The experimental data shown in FIGS. 8a and 8b illustrate that limit of detection for trypsin using the present gold nanoparticle-based colorimetric assay could be further improved by using gold nanoparticles with larger average diameter of 30 nm compared to the results obtained with gold nanoparticles having an average diameter of 20 nm. FIG. 8a displays the ultraviolet-visible spectra of solutions of colloidal gold nanoparticles after being added to solutions of the peptide substrate SEQ ID NO: 1 Lys-Lys-Gly-Phe-Pro-Arg-Gly-Gly-Asp-Cys which were pre-exposed to trypsin at different concentrations of up to 200 pM for 3 hours at a temperature of 20° C. The concentration of peptide substrate used was 750 nM and the negatively charged gold nanoparticles used had an average diameter of 30 nm. The results displayed in FIG. 8a are summarized in FIG. 8b, which displays the ratio of the absorbance at 650 nm to the absorbance at 520 nm of the solutions of colloidal gold nanoparticles after being added to the solutions of the peptide substrate SEQ ID NO: 1 Lys-Lys-Gly-Phe-Pro-Arg-Gly-Gly-Asp-Cys pre-exposed to trypsin for 3 hours as function of the concentration of trypsin. It is shown that by increasing the average diameter of the gold nanoparticles from 20 nm to 30 nm, the limit of detection of trypsin using the colloidal gold nanoparticle-based colorimetric assay developed in the present invention was further improved from about 2 to 10 pM to a range of from 500 fM to 2 pM.

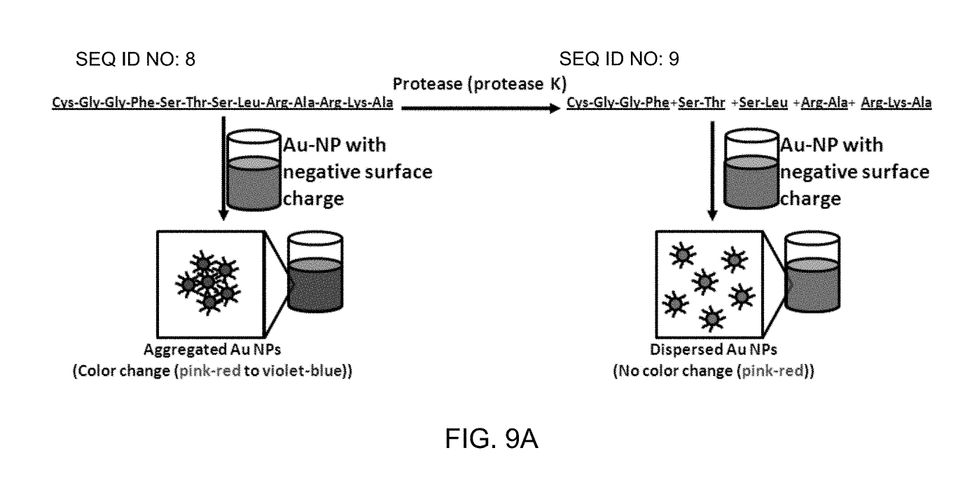

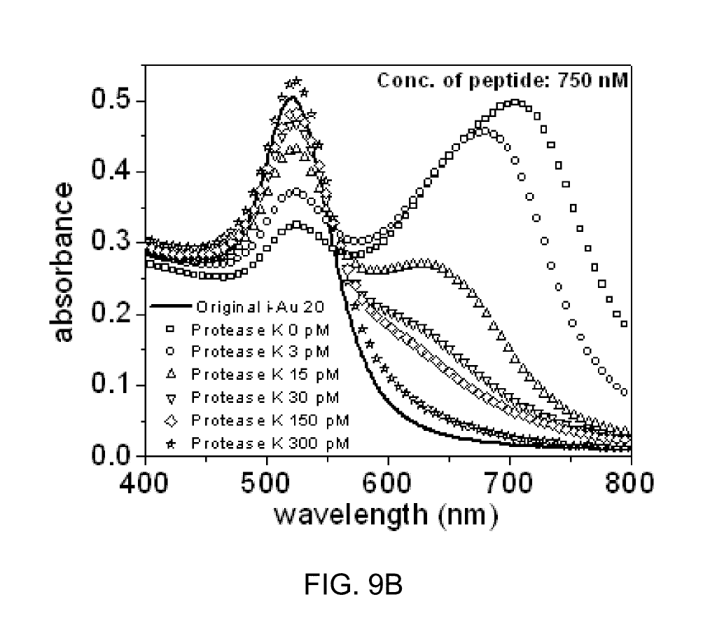

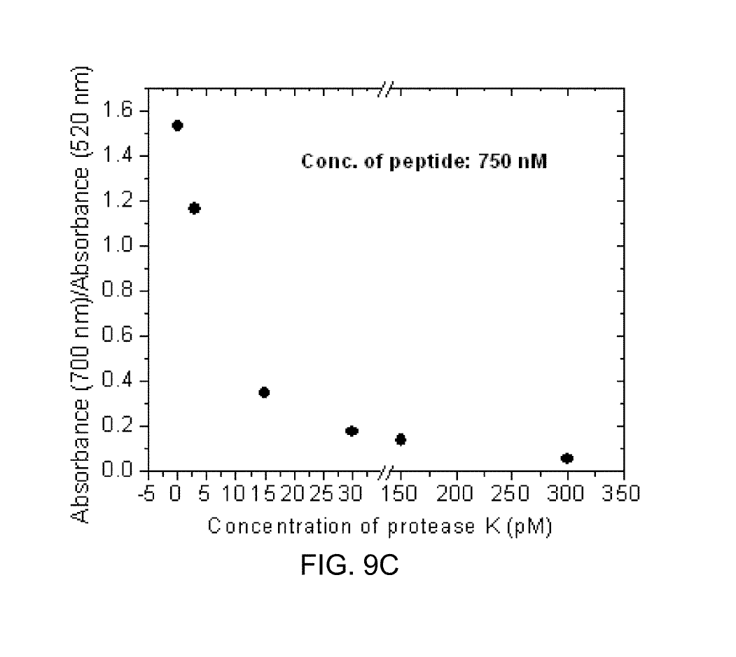

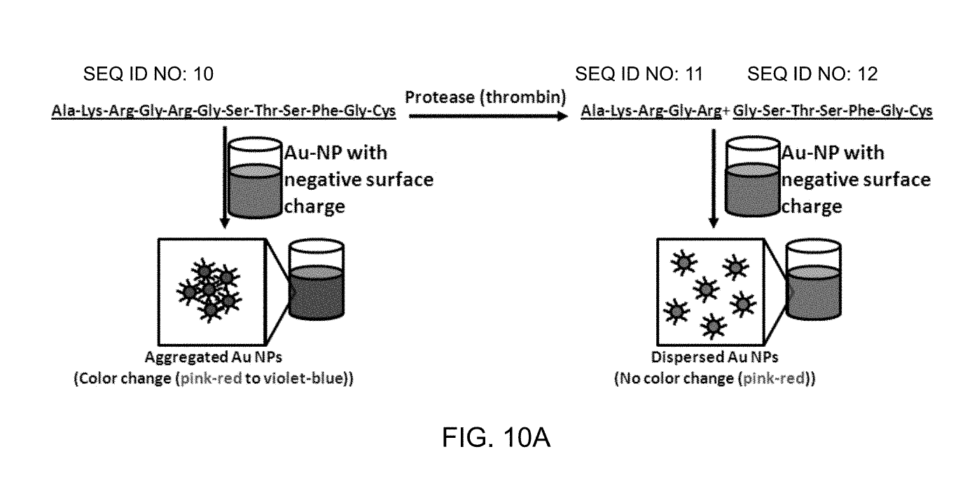

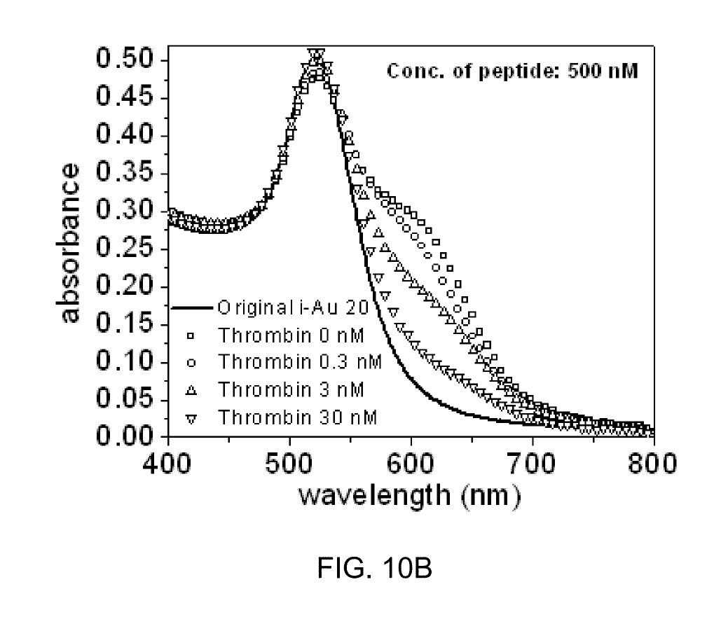

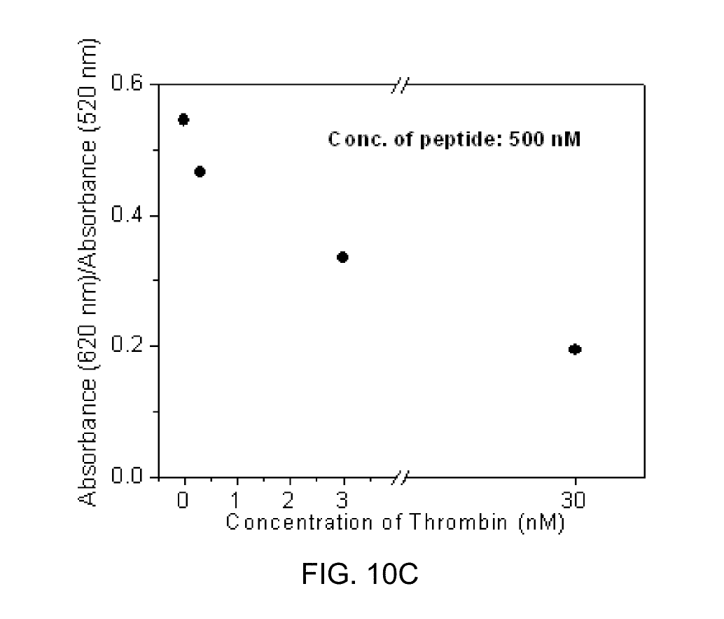

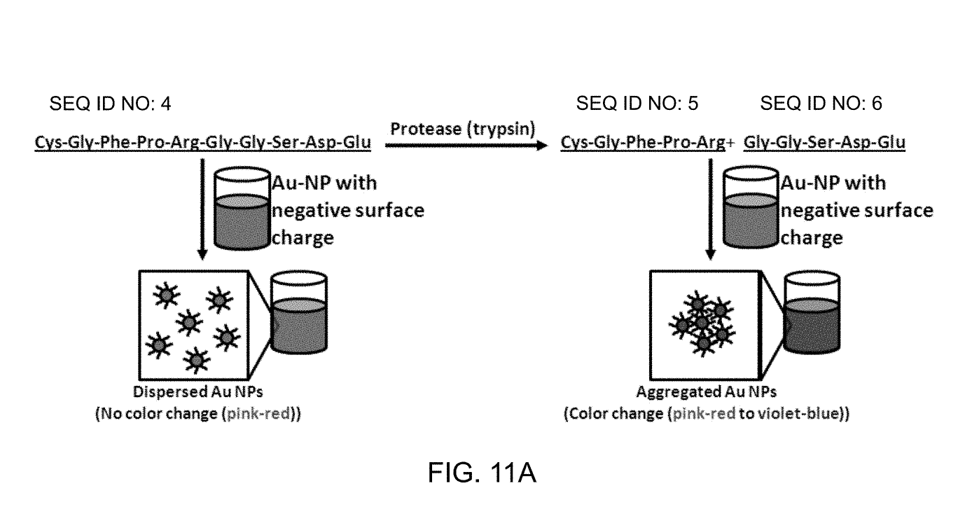

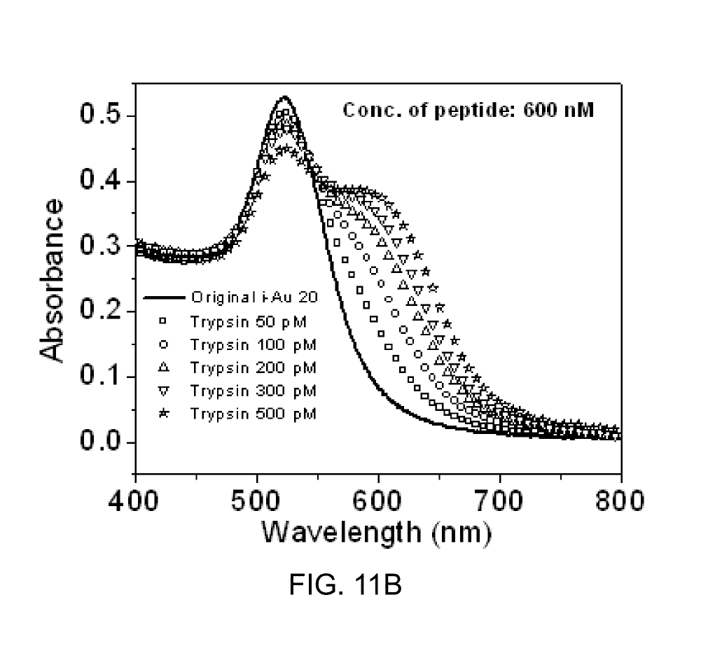

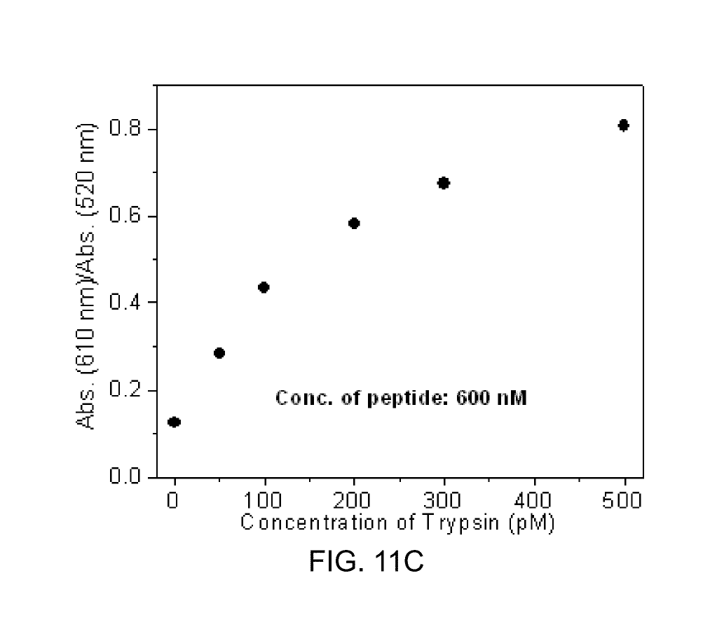

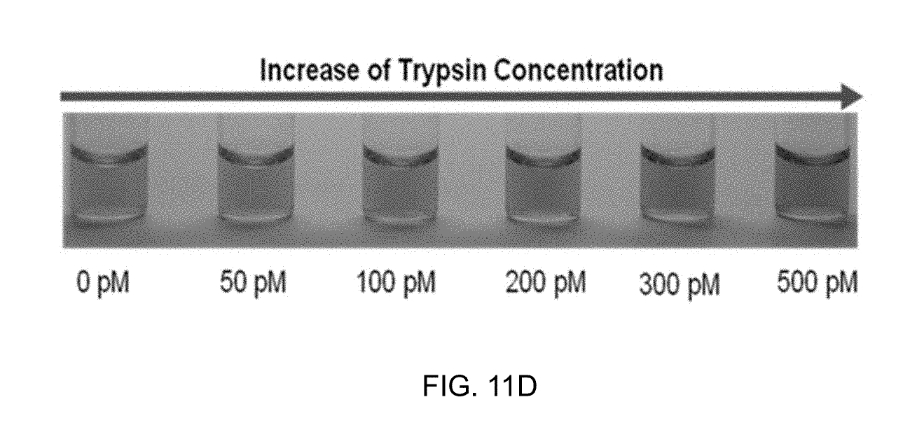

In the experiments described above in this specification, the detection strategy shown in the FIG. 1a was used to detect trypsin activity using the colloidal gold nanoparticle-based colorimetric assay of the present invention and this strategy was chosen for illustration purposes only. The present invention is not limited to using only the detection strategy shown in FIG. 1a. The other three detection strategies shown in FIGS. 1b, 1c, and 1d, respectively can also be used for the detection of protease activity using the present colloidal gold nanoparticle-based colorimetric assay. An example of use of the scheme of FIG. 1b is shown below in FIGS. 11a to 11d. In addition, the assay is easily adaptable for detection of other proteases as shown from the data of FIGS. 9 and 10, discussed below.

The assay can be readily adapter for use with other proteases, such as protease K. Protease K has a broad ability to degrade proteins, the main site of cleavage is the peptide bond adjacent to the carboxyl group of aliphatic and aromatic amino acids with blocked alpha amino groups. The peptide substrate used to test the assay for its ability to detect protease K activity is shown in FIG. 9a. The gold nanoparticles used had an average diameter of 20 nm and a negative surface charge. The peptide substrate had the ability to cause aggregation of the gold nanoparticles in its intact state. The crosslinking reaction is believed to occur by a covalent bond of a first gold nanoparticle to the amino acid Cys on the amino end of the peptide substrate. The ionic bonds with a second gold nanoparticle are the result of the positive charges from the Arg, and Lys amino acids near the carboxy end of the peptide substrate. The results demonstrate that the ionic bond forming amino acids do not have to be on the amino or carboxy terminal ends, they can be interior and still function to cause aggregation. Once the protease K completely cleaves the peptide substrate one expects the hydrolytic fragments shown in FIG. 9a. As can be seen after cleavage the Cys is no longer connected in the peptide substrate to the Arg or Lys amino acids and thus crosslinking is lost. FIG. 9b shows the UV-VIS absorption spectra for the native gold nanoparticles, the spectrum caused by the presence of 750 nM of peptide substrate and the spectrum for a series of peptide substrates pre-exposed to a series of protease K concentrations. The data shows that the aggregation in the presence of 750 nM peptide substrate shifts the absorption maximum to a new value of 700 nm from the initial value of 520 nm±5 nm and the ratio of the absorption at 700 nm/520 nm is approximately 1.54. With increasing amounts of protease K the spectrum shifts back toward the native spectrum seen in the gold nanoparticles alone and the ratio falls to nearly 0 as shown in FIGS. 9 b and 9c. The results demonstrate the ability to adapt the assay to use with other proteases and the ability to obtain rapid qualitative and quantitative data for protease activity. An inhibitor of protease K could be measured in the same assay by adding the step of pre-incubating the protease K with the inhibitor solution prior to the pre-exposure of the peptide substrate. Inhibitor activity would result in less of a shift in the absorption spectrum and a higher ratio of 700 nm/520 nm for the same level of protease K.

FIGS. 10a to 10c show the data from use of the assay, scheme 1a again, to measure the activity of the protease thrombin. The actual cleavage site for thrombin is the sequence SEQ ID NO: 7 Leu-Val-Pro-Arg-Gly-Ser with the cleavage occurring between the Arg and Gly. As shown in FIG. 10a the peptide substrate we used had the central portion of this sequence, namely Arg-Gly-Ser. As shown the native peptide sequence was able to crosslink and aggregate the gold nanoparticles, although the aggregation shift was not as large as for other peptide substrates. The shift with aggregation caused appearance of a new absorption peak at 620 nm. As shown in FIGS. 10b and 10c the activity of the thrombin could be followed by monitoring the ratio of the absorbance at 620 nm/520 nm.

For the detection strategy shown in FIG. 1b, colloidal gold nanoparticles with a negative surface charge are used in the detection of trypsin activity and the intact peptide substrates could not induce the aggregation of the negatively charged gold nanoparticles. The peptide substrate had an amino acid sequence of SEQ ID NO: 4 Cys-Gly-Phe-Pro-Arg-Gly-Gly-Ser-Asp-Glu and there was no color change of the solution of colloidal gold nanoparticles when the intact peptide substrate was added to the negatively charged gold nanoparticles. Following on the theory of the present invention, this peptide substrate can covalently bond to the gold nanoparticles via a thiol bond with the terminal Cys; however the other end of the peptide substrate has two negative charges from the Asp and Glu and thus the nanoparticles with bound peptide are even more repelled from each other than the native nanoparticles. After hydrolytic cleavage of the original peptide substrate by the protease analyte, for this example trypsin, the hydrolyzed peptide fragments could cross-link the negatively charged gold nanoparticles, leading to color changes of the solution of colloidal gold nanoparticles from pink-red to violet-blue. This is accompanied by a shift in the absorbance band maximum from 520 nm±5 nm to 610 nm. Following complete cleavage the fragment SEQ ID NO: 5 Cys-Gly-Phe-Pro-Arg can bond to the gold nanoparticles both through the Cys and via an ionic bond through the positively charged amino acid Arg. The data of FIGS. 11a and 11c show the shifts in absorption spectra and ratio of 610/520 caused by the hydrolytic cleavage. FIG. 11d is a series of photographs showing the visual color changes that accompany the aggregation by the hydrolytic fragment.

For the detection strategy shown in FIG. 1c, colloidal gold nanoparticles with a positive surface charge are used in the detection and the original peptide substrates induce the aggregation of the positively charged gold nanoparticles, the peptide substrate could have an amino acid sequence of SEQ ID NO: 4 Cys-Gly-Phe-Pro-Arg-Gly-Gly-Ser-Asp-Glu, leading to a color change of the solution of colloidal gold nanoparticles from pink-red to violet-blue by the intact peptide. The bonds are through the terminal Cys covalent linkage and ionically through the terminal Asp and Glu. After hydrolytic cleavage of the original peptide substrates by the protease analyte, for this example trypsin, the hydrolyzed peptide fragments could not cross-link the positively charged gold nanoparticles and no change of the color of the solution of colloidal gold nanoparticles would be observed. For the detection strategy shown in FIG. 1d, colloidal gold nanoparticles with a positive surface charge are used in the detection and the original peptide substrates, with an amino acid sequence of SEQ ID NO: 1 Lys-Lys-Gly-Phe-Pro-Arg-Gly-Gly-Asp-Cys for example could not induce the aggregation of the positively charged gold nanoparticles, resulting in no color change of the solution of colloidal gold nanoparticles. While the Cys could bond to the gold nanoparticles the terminal Lys will repel other positively charged nanoparticles. After hydrolytic cleavage of the original peptide substrates by a protease analyte, for this example trypsin, the hydrolyzed peptide fragments could cross-link the positively charged gold nanoparticles, leading to a color change of the solution of colloidal gold nanoparticles from pink-red to violet-blue. Other proteases will likely required different peptide sequences from those shown, the peptide substrate sequences shown are for illustrative purposes only, and the invention is not limited to these sequences.

Based on the observations in our studies, we believe the discussed detection strategies of the colloidal gold nanoparticle-based assay shown in the FIGS. 1a, 1b, 1c, and 1d are of a general nature and could also be used for the detection of protease inhibitors. In this use of the assay procedure one prepares the standard curves using samples of gold nanoparticles combined with the peptide substrate pre-exposed to various levels of the protease. Then the protease inhibitor is detected by observing the changes to the same standard curve run in the presence of samples suspected to contain the protease inhibitor. If the inhibitor is present then the measured changes should approach what is found by combining the intact peptide substrate with the gold nanoparticles in the absence of any protease. So if for illustration purposes we chose a peptide substrate wherein the intact peptide substrate and not its hydrolytic fragments causes aggregation of the gold nanocolloids as seen by a color change from pink-red to violet-blue and a shift of the absorbance band maximum from 520 nm±5 nm toward say 650 nm for 20 nm nanoparticles; then the presence of the protease will cause a reduction in the level of aggregation normally caused by the peptide substrate. A standard curve is prepared by pre-exposing a constant level of peptide substrate to a series of protease concentrations at a given predetermined temperature and for a predetermined amount for time and then adding the pre-exposed solution to the gold nanoparticles and measuring the absorbance spectra. Then a liquid, which may be a biological fluid, suspected of containing the protease inhibitor is mixed with an aqueous solution of the protease to generate a solution containing both the protease and the protease inhibitor. The liquid may be any biological fluid or an extract from a biological tissue. The solution of protease and potential protease inhibitor are incubated at a predetermined temperature, generally a temperature of above about 20 degrees Celsius, for a sufficient amount of time, for example a time of between 0.5 to 3 hours, to enable said protease inhibitor to block the hydrolytic function of the protease. In the next step an aqueous solution of the peptide substrate specific for the protease is added to the solution to generate a mixture containing the protease, the protease inhibitor, and the peptide substrate. The mixture is then incubated at a predetermined temperature for a sufficient amount of time, for example a time between 0.5 to 3 hours, to enable some hydrolytic cleavage of the peptide substrate by the protease. Then a colloidal suspension of gold nanoparticles with a pink-red color, meaning non-aggregated, is added to the mixture and incubated for period of time of 30 minutes or less. The state of aggregation is then compared to the aggregation caused in the presence of the same protocol done in the absence of the suspected protease inhibitor. The amount of the peptide remaining uncleaved, which depends on the amount of the protease inhibitor in the liquid, determines the state of aggregation of the gold nanoparticles that is induced, resulting in the formation of a final solution with characteristic color in 30 minutes or less. Next qualitatively detecting the amount of the protease inhibitor in the liquid by comparing the characteristic color of the final solution with the colors of standard samples containing known amounts of the protease inhibitor and quantitatively detecting the amount of protease inhibitor in the biological fluid by examining the ultraviolet-visible spectrum of the final solution and comparing the ratio of the absorbance at the new maximum, in this example 650 nm, to the absorbance at 520 nm±5 nm of the final solution to ratios of the absorbance at 650 nm to the absorbance at 520 nm of standard samples containing known amounts of protease inhibitor. A similar protocol would be followed for the examples wherein a hydrolytic fragment causes the aggregation and not the intact peptide as described above.

Generally to achieve the best results in this sort of a scheme the levels of peptide substrate and gold nanoparticles are selected to cause near maximal aggregation of the gold nanoparticles by the level of peptide substrate. Then several levels of protease are chosen to give a range of cleavage, generally so that near maximal cleavage occurs at the highest level of the protease and using the results from the presence and absence of the protease gives the widest range for the standards. Then several levels of suspected protease inhibitor are chosen for each level of protease so that the results with one or more of the inhibitor levels fall within the range of the standards. As would be understood after the above explanation the inventive assay methods of the present invention can also be used to detect new previously unknown protease inhibitors by determining the effect of adding a series of levels of the liquid containing the suspected protease inhibitor to a standard curve of the protease and peptide substrate on the measured aggregation state of the gold nanoparticles.

Protease inhibitors of special interest which could be detected by the colloidal gold nanoparticle-based colorimetric assay developed in the present invention comprise, but are not limited to, saquinavir, ritonavir, indinavir, nelfinavir, amprenavir, boceprevir, telaprevir, bovine pancreatic trypsin inhibitor, serine protease inhibitor Kazal-type 1, and Alpha-1 antitrypsin.

The present assay methods can also be used to probe the active sites of proteases by taking a known peptide substrate, altering the proposed recognition sequence or amino acid sequence on either side of a cleavage site and determining how this effects the ability to cleave the peptide substrate as seen by changes in the measured aggregation state of the gold nanoparticles. In addition, the assay method can be used to determine cleavage sites by altering the amino acid sequence and determining the effect on the measured aggregation state of the gold nanoparticles.

In the experiments described in this specification, deionized water was selected as the liquid medium for the gold nanoparticles, protease, protease inhibitor, peptide substrate and the assay process. However, other more biological fluids can also be used as dissolution media for any of these components and for the reactions. For example, biological fluids can be chosen from, but not limited to, blood, plasma, saliva, urine, distilled water, a phosphate buffer saline (PBS) solution, a buffer for High Performance Capillary Electrophoresis, a hydroxyethyl piperazineethanesulfonic acid (HEPES) sodium salt solution, a citrate-phosphate-dextrose solution, a phosphate buffer solution, a sodium acetate solution, a sodium chloride solution, a sodium DL-lactate solution, a tris(hydroxymethyl) aminomethane ethylenediaminetetraacetic acid (Tris-EDTA) buffer solution, a tris(hydroxymethyl) aminomethane (Tris) buffered saline, or mixtures thereof. For some of the biological fluids, such as serum, one may have to engage in some pre-purification to remove serum proteins which can themselves cause aggregation of the gold nanoparticles. In some cases it may be necessary to extract the proteases or protease inhibitors from the original biological fluids prior to analysis in the present assay systems.

In the experiments described in this specification, gold nanoparticles used in the experiments are spherical gold nanoparticles with an average diameter of 20 or 30 nm. However, colloidal gold nanoparticles with other shapes and configurations, including rods, prisms, disks, cubes, core-shell structures, cages, and frames, wherein they have at least one dimension in the range of from 1 to 200 nm, could also work for the colloidal gold nanoparticle-based colorimetric assay developed in the present invention for the detection of the proteases and protease inhibitors. In addition, nanostructures which have outer surfaces that are only partially covered with gold should also work for the gold colorimetric assay developed in the present invention.

Although the described process of fabrication of colloidal gold nanoparticles by laser ablation of bulk gold target in a colloidal suspension liquid was illustrated in embodiments wherein the liquid was deionized water, it is possible to carry out the processes described in other liquids. For example, laser ablation of a bulk gold target can be carried out in water, methanol, ethanol, acetone, and other organic solvents.

In the experiments described in this specification, the mixtures were incubated at a temperature of approximately 20° C. In principle this temperature could vary between about 20 to 50° Celsius for optimizing the efficiency of the hydrolytic cleavage of the peptide substrate by the protease when the protease is any other mammalian enzyme. As discussed above temperature ranges outside this range can be used when assays are conducted on proteases from either heat-tolerant or cold-tolerant sources. The same considerations apply to use of the present assay methods for determination of protease inhibitor activity.

In an embodiment the present invention is a method for detecting hydrolytic activity of a protease comprising the following steps: providing a solution of non-aggregated colloidal gold nanoparticles, the colloidal gold nanoparticles having a surface charge and a property of a visible pink-red color when non-aggregated and a visible violet-blue color when aggregated; providing a peptide substrate having the following properties: at least one first functional group that bonds to the gold nanoparticles independently of the surface charge on the gold nanoparticles, at least one second functional group that can form an ionic bond with the surface charge of the gold nanoparticles, located between the first functional group and the second functional group an amino acid sequence having at least one cleavage site for the protease, wherein the peptide substrate aggregates the gold nanoparticles when intact through crosslinking of the gold nanoparticles by the first and the second functional groups and does not aggregate the gold nanoparticles following cleavage by the protease at the cleavage site; forming a first mixture by adding to a solution of the gold nanoparticles a solution of the peptide substrate and detecting a color change in the first mixture indicative of aggregation of the gold nanoparticles; forming a reaction mixture by adding to a solution of the peptide substrate a solution of the protease and incubating the reaction mixture at a predetermined temperature for a predetermined amount of time, wherein the predetermined temperature and the predetermined time are sufficient to permit cleavage of the cleavage site in the peptide substrate by the protease; forming a second mixture by adding to a solution of the gold nanoparticles the reaction mixture after incubation and detecting a color change in the second mixture indicative of aggregation of the gold nanoparticles; and comparing the color of the second reaction mixture to the color of the first mixture to determine a reduction in aggregation of the gold nanoparticles indicative of proteolytic activity of the protease on the peptide substrate.

In one or more embodiments the present invention comprises providing gold nanoparticles have at least one dimension in the range of from 1 to 200 nanometers.

In one or more embodiments the present invention comprises providing gold nanoparticles having a shape selected from the group consisting of a sphere, a rod, a prism, a disk, a cube, a core-shell structure, a cage, a frame, or a mixture thereof.

In one or more embodiments the present invention comprises providing a solution of gold nanoparticles suspended in one of water, methanol, ethanol, acetone, a biological buffer, or a mixture thereof.

In one or more embodiments the present invention comprises providing the gold nanoparticles as a nanostructure at least partially covered by said gold nanoparticles.

In one or more embodiments the present invention comprises providing a peptide substrate having as the first functional group a thiol group, an amine group, a phosphine group, a disulfide group, or a mixture thereof.

In one or more embodiments the present invention comprises providing a peptide substrate wherein the first functional group comprises at least one of a cysteine, a methionine, or a mixture thereof.

In one or more embodiments the present invention comprises providing a peptide substrate wherein the second functional group comprises at least one of an arginine, a lysine, a histidine, an aspartic acid, a glutamic acid, or a mixture thereof.

In one or more embodiments the present invention comprises providing gold nanoparticles having a negative surface charge and further comprises providing a peptide substrate wherein the second functional group comprises at least one of an arginine, a lysine, a histidine, or a mixture thereof.

In one or more embodiments the present invention comprises providing gold nanoparticles having a positive surface charge and further comprises providing a peptide substrate wherein the second functional group comprises at least one of an aspartic acid, a glutamic acid, or a mixture thereof.

In one or more embodiments the present invention comprises providing a peptide substrate having a cleavage site cleaved by at least one of a serine protease, a threonine protease, a cysteine protease, an aspartate protease, a glutamic acid protease, a metalloprotease, or a mixture thereof.

In one or more embodiments the present invention comprises providing a peptide substrate having a cleavage site cleaved by at least one of trypsin, chymotrypsin, thrombin, prostrate-specific antigen, HIV-1 protease, elastase, a metalloendopeptidase, subtilisin, or a mixture thereof.

In one or more embodiments the present invention comprises incubating the reaction mixture at a temperature of from 10° C. to 65° C. for a period of time of from 10 minutes to 3 hours.

In one or more embodiments the present invention comprises visually detecting a color change in the first and the second mixtures and further comprises comparing the color of the second mixture to the color of the first mixture visually.

In one or more embodiments the present invention comprises detecting a color change in the first and the second mixtures by measuring an ultraviolet-visible spectrum of the first and the second mixtures and further comprises comparing the spectrum of the second mixture to the spectrum of the first mixture.

In one or more embodiments the present invention comprises preparing an aggregation standard curve by adding a series of different peptide substrate concentrations to a series of gold nanoparticles each at the same concentration and further comprises comparing the color of the second reaction mixture to the standard curve to determine the reduction of aggregation and to determine quantitatively a proteolytic activity of the protease.

In one or more embodiments the present invention comprises visually detecting a color change in the first and the second mixtures and further comprises comparing the color of the second mixture to the colors of the standard curve visually.

In one or more embodiments the present invention comprises detecting a color change in the first and the second mixtures by measuring an ultraviolet-visible spectrum of the first and the second mixtures and further comprises comparing the spectrum of the second mixture to the spectra of the standard curve.

In one or more embodiments of the present invention the solution of the protease comprises one of deionized water, distilled water, phosphate buffered saline, a High Performance Capillary Electrophoresis solution, a 4-(2-hydroxyethyl)-1-piperazineethanesulfonic acid buffer (HEPES) solution, a citrate-phosphate-dextrose solution, a phosphate buffer, a sodium acetate solution, a sodium chloride solution, a sodium DL-lactate solution, a tris(hydroxymethyl) aminoethane ethylenediaminetetraacetic acid Tris-EDTA buffer solution, a tris(hydroxymethyl) aminomethane (Tris) buffered saline solution, blood, plasma, saliva, urine, or a mixture thereof.

In one or more embodiments the present invention comprises the further steps of detecting an inhibitor of the protease by incubating a solution of the protease inhibitor with the solution of the protease at a predetermined temperature for a predetermined period of time to form an inhibited protease solution, then adding the inhibited protease solution as the solution of the protease and then comparing the color of the second reaction mixture formed from the inhibited protease solution to a second reaction mixture formed with the same amount of the protease that was not incubated with the protease inhibitor to detect a decrease in the reduction in aggregation caused by the protease.

In one embodiment the present invention is a method for detecting hydrolytic activity of a protease comprising the following steps: providing a solution of non-aggregated colloidal gold nanoparticles, the colloidal gold nanoparticles having a surface charge and a property of a visible pink-red color when non-aggregated and a visible violet-blue color when aggregated; providing a peptide substrate having the following properties: at least one first functional group that bonds to the gold nanoparticles independently of the surface charge on the gold nanoparticles, at least one second functional group that is capable of forming an ionic bond with the surface charge of the gold nanoparticles, located outside the peptide sequence between the first functional group and the second functional group an amino acid sequence having at least one cleavage site for the protease, wherein the peptide substrate does not aggregate the gold nanoparticles when intact and wherein at least one proteolytic fragment containing the first functional group and the second functional group does aggregate the gold nanoparticles following cleavage by the protease at the cleavage site; forming a first mixture by adding to a solution of the gold nanoparticles a solution of the peptide substrate and detecting a color of the first mixture indicative of non-aggregation of the gold nanoparticles; forming a reaction mixture by adding to a solution of the peptide substrate a solution of the protease and incubating the reaction mixture at a predetermined temperature for a predetermined amount of time, wherein the predetermined temperature and the predetermined time are sufficient to permit cleavage of the cleavage site in the peptide substrate by the protease; forming a second mixture by adding to a solution of the gold nanoparticles the reaction mixture after incubation and detecting a color change in the second mixture indicative of aggregation of the gold nanoparticles; and comparing the color of the second reaction mixture to the color of the first mixture to determine a degree of aggregation of the gold nanoparticles indicative of proteolytic activity of the protease on the peptide substrate.

In one or more embodiments of the present invention the protease inhibitor comprises at least one of saquinavir, ritonavir, indinavir, nelfinavir, amprenavir, boceprevir, telaprevir, bovine pancreatic trypsin inhibitor, serine protease inhibitor Kazal-type 1, Alpha-1 antitrypsin, and mixtures thereof.

In one or more embodiments the present invention is an assay kit for detecting proteolytic activity of a protease comprising: a plurality of non-aggregated gold nanoparticles, each of the gold nanoparticles having a surface charge; a peptide substrate comprising a peptide sequence, the peptide substrate comprising: at least one first functional group capable of covalently bonding to one of the gold nanoparticles; at least one second functional group capable of forming an ionic bond with the surface charge of one of the gold nanoparticles; and a cleavage site for the protease, the cleavage site at a location either in a peptide sequence between the first and the second functional groups or outside the peptide sequence between the first and the second functional groups, wherein when the cleavage site is at a location in the peptide sequence between the first and the second functional groups then the peptide substrate is capable of crosslinking the gold nanoparticles and aggregating the nanoparticles and no hydrolytic fragments of the peptide substrate formed following cleavage of the peptide substrate by the protease are capable of crosslinking the gold nanoparticles, and wherein when the cleavage site is at a location outside the peptide sequence between the first and the second functional groups then the peptide substrate is not capable of crosslinking the gold nanoparticles and cannot aggregate the nanoparticles and a hydrolytic fragment containing the first and the second functional groups is capable of crosslinking the gold nanoparticles after formation following cleavage of the peptide substrate by the protease and the hydrolytic fragment causes aggregation of the gold nanoparticles; and the non-aggregated gold nanoparticles having an absorbance spectrum with a maximum absorbance peak at 520 nanometers±5 nanometers, wherein a solution of the non-aggregated gold nanoparticles has a visible color of pink-red; and wherein aggregation of the gold nanoparticles causes a shift in the absorbance spectrum with formation of a new absorbance peak in the range of from 600 nanometers to 750 nanometers and a decrease of the absorbance at 520 nanometers±5 nanometers, the shift leading to a change in the visible color to a violet-blue color.

In one or more embodiments the present invention comprises gold nanoparticles have a size in at least one dimension of from 1 to 200 nanometers.

In one or more embodiments the present invention comprises gold nanoparticles have a shape selected from the group consisting of a sphere, a rod, a prism, a cube, a disk, a core-shell structure, a frame, a cage, a mixture thereof.

In one or more embodiments the present invention comprises gold nanoparticles provided as a solution in one of water, methanol, ethanol, acetone, a biological buffer, or a mixture thereof.

In one or more embodiments the present invention comprises gold nanoparticles provided as a nanostructure at least partially covered by the gold nanoparticles.

In one or more embodiments of the present invention the at least one first functional group comprises a thiol group, an amine group, a phosphine group, a disulfide group or a mixture thereof.

In one or more embodiments of the present invention the at least one first functional group comprises a cysteine, a methionine, or a mixture thereof.

In one or more embodiments of the present invention the at least one second functional group comprises arginine, lysine, histidine, aspartic acid, glutamic acid, or a mixture thereof.

In one or more embodiments the present invention comprises gold nanoparticles wherein the surface charge is negative and the at least one second functional group is selected from the group consisting of lysine, arginine, histidine, and mixtures thereof.

In one or more embodiments the present invention comprises gold nanoparticle wherein the surface charge is positive and the at least one second functional group is selected from the group consisting of aspartic acid, glutamic acid, and mixtures thereof.

In one or more embodiments the present invention comprises a peptide substrate wherein the cleavage site is cleaved by at least one of a serine protease, a threonine protease, a cysteine protease, an aspartate protease, a glutamic acid protease, a metalloprotease, or a mixture thereof.

In one or more embodiments the present invention comprises a peptide substrate wherein the cleavage site is cleaved by at least one of trypsin, chymotrypsin, thrombin, prostrate-specific antigen, HIV-1 protease, elastase, a metalloendopeptidase, subtilisin, or a mixture thereof.

In one or more embodiments the present invention further comprises a buffer solution comprising deionized water, distilled water, phosphate buffered saline, a High Performance Capillary Electrophoresis solution, a 4-(2-hydroxyethyl)-1-piperazineethanesulfonic acid buffer (HEPES) solution, a citrate-phosphate-dextrose solution, a phosphate buffer, a sodium acetate solution, a sodium chloride solution, a sodium DL-lactate solution, a tris(hydroxymethyl) aminoethane ethylenediaminetetraacetic acid Tris-EDTA buffer solution, a tris(hydroxymethyl) aminomethane (Tris) buffered saline solution, or a mixture thereof.

In one embodiment the present invention is an assay kit for detecting inhibition of a proteolytic activity of a protease comprising: a protease; a plurality of non-aggregated gold nanoparticles, each of the gold nanoparticles having a surface charge; a peptide substrate comprising a peptide sequence, the peptide substrate comprising: at least one first functional group capable of covalently bonding to one of the gold nanoparticles; at least one second functional group capable of forming an ionic bond with the surface charge of one of the gold nanoparticles; and a cleavage site for the protease, the cleavage site at a location either in a peptide sequence between the first and the second functional groups or outside the peptide sequence between the first and the second functional groups, wherein when the cleavage site is at a location in the peptide sequence between the first and the second functional groups then the peptide substrate is capable of crosslinking the gold nanoparticles and aggregating the nanoparticles and no hydrolytic fragments of the peptide substrate formed following cleavage of the peptide substrate by the protease are capable of crosslinking the gold nanoparticles, and wherein when the cleavage site is at a location outside the peptide sequence between the first and the second functional groups then the peptide substrate is not capable of crosslinking the gold nanoparticles and cannot aggregate the nanoparticles and a hydrolytic fragment containing the first and the second functional groups is capable of crosslinking the gold nanoparticles after formation following cleavage of the peptide substrate by the protease and the hydrolytic fragment causes aggregation of the gold nanoparticles; and the non-aggregated gold nanoparticles having an absorbance spectrum with a maximum absorbance peak at 520 nanometers±5 nanometers, wherein a solution of the non-aggregated gold nanoparticles has a visible color of pink-red; and wherein aggregation of the gold nanoparticles causes a shift in the absorbance spectrum with formation of a new absorbance peak in the range of from 600 nanometers to 750 nanometers and a decrease of the absorbance at 520 nanometers±5 nanometers, the shift leading to a change in the visible color to a violet-blue color.

In one or more embodiments of the present invention the protease inhibitor comprises at least one of saquinavir, ritonavir, indinavir, nelfinavir, amprenavir, boceprevir, telaprevir, bovine pancreatic trypsin inhibitor, serine protease inhibitor Kazal-type 1, Alpha-1 antitrypsin, or mixtures thereof.

It is intended that the invention be limited only by the claims which follow, and not by the specific embodiments and their variations and combinations as described herein-above.

What is claimed is:

1. A method for detecting hydrolytic activity of a protease comprising the following steps:
a) providing a solution of non-aggregated colloidal gold nanoparticles, said colloidal gold nanoparticles having a surface charge and a property of a visible pink-red color when non-aggregated and a visible violet-blue color when aggregated;
b) providing a peptide substrate having the following properties: at least one first functional group that bonds to said gold nanoparticles independently of the surface charge on said gold nanoparticles, at least one second functional group that can form an ionic bond with the surface charge of said gold nanoparticles, located between said first functional group and said second functional group an amino acid sequence having at least one cleavage site for said protease, wherein said peptide substrate aggregates said gold nanoparticles when intact through crosslinking of said gold nanoparticles by said first and said second functional groups and does not aggregate said gold nanoparticles following cleavage by said protease at said cleavage site;
c) forming a first mixture by adding to a solution of said gold nanoparticles a solution of said peptide substrate and detecting a color change in said first mixture indicative of aggregation of said gold nanoparticles;
d) forming a reaction mixture by adding to a solution of said peptide substrate a solution of said protease and incubating said reaction mixture at a predetermined temperature for a predetermined amount of time, wherein said predetermined temperature and said predetermined time are sufficient to permit cleavage of said cleavage site in said peptide substrate by said protease;
e) forming a second mixture by adding to a solution of said gold nanoparticles said reaction mixture after incubation in step d) and detecting a color change in said second mixture indicative of aggregation of said gold nanoparticles; and
f) comparing the color of said second reaction mixture to the color of said first mixture to determine a reduction in aggregation of said gold nanoparticles indicative of proteolytic activity of said protease on said peptide substrate.

2. A method for detecting hydrolytic activity of a protease comprising the following steps:
a) providing a solution of non-aggregated colloidal gold nanoparticles, said colloidal gold nanoparticles having a surface charge and a property of a visible pink-red color when non-aggregated and a visible violet-blue color when aggregated;
b) providing a peptide substrate having the following properties: at least one first functional group that bonds to said gold nanoparticles independently of the surface charge on said gold nanoparticles, at least one second functional group that is capable of forming an ionic bond with the surface charge of said gold nanoparticles, located outside the peptide sequence between said first functional group and said second functional group an amino acid sequence having at least one cleavage site for said protease, wherein said peptide substrate does not aggregate said gold nanoparticles when intact and wherein at least one proteolytic fragment containing said first functional group and said second functional group does aggregate said gold nanoparticles following cleavage by said protease at said cleavage site;
c) forming a first mixture by adding to a solution of said gold nanoparticles a solution of said peptide substrate and detecting a color of said first mixture indicative of non-aggregation of said gold nanoparticles;
d) forming a reaction mixture by adding to a solution of said peptide substrate a solution of said protease and incubating said reaction mixture at a predetermined temperature for a predetermined amount of time, wherein said predetermined temperature and said predetermined time are sufficient to permit cleavage of said cleavage site in said peptide substrate by said protease;
e) forming a second mixture by adding to a solution of said gold nanoparticles said reaction mixture after incubation in step d) and detecting a color change in said second mixture indicative of aggregation of said gold nanoparticles; and
f) comparing the color of said second reaction mixture to the color of said first mixture to determine a degree of aggregation of said gold nanoparticles indicative of proteolytic activity of said protease on said peptide substrate.

3. An assay kit for detecting proteolytic activity of a protease comprising:
a) a plurality of non-aggregated gold nanoparticles, each of said gold nanoparticles having a surface charge;
b) a peptide substrate comprising a peptide sequence, said peptide substrate comprising: at least one first functional group capable of covalently bonding to one of said gold nanoparticles; at least one second functional group capable of forming an ionic bond with said surface charge of one of said gold nanoparticles; and a cleavage site for said protease, said cleavage site at a location either in a peptide sequence between said first and said second functional groups or outside said peptide sequence between said first and said second functional groups, wherein when said cleavage site is at a location in said peptide sequence between said first and said second functional groups then said peptide substrate is capable of crosslinking said gold nanoparticles and aggregating said nanoparticles and no hydrolytic fragments of said peptide substrate formed following cleavage of said peptide substrate by said protease are capable of crosslinking said gold nanoparticles, and wherein when said cleavage site is at a location outside said peptide sequence between said first and said second functional groups then said peptide substrate is not capable of crosslinking said gold nanoparticles and cannot aggregate said nanoparticles and a hydrolytic fragment containing said first and said second functional groups is capable of crosslinking said gold nanoparticles after formation following cleavage of said peptide substrate by said protease and said hydrolytic fragment causes aggregation of said gold nanoparticles;
c) said non-aggregated gold nanoparticles having an absorbance spectrum with a maximum absorbance peak at 520 nanometers±5 nanometers, wherein a solution of said non-aggregated gold nanoparticles has a visible color of pink-red; and wherein aggregation of said gold nanoparticles causes a shift in said absorbance spectrum with formation of a new absorbance peak in the range of from 600 nanometers to 750 nanometers and a decrease of said absorbance at 520 nanometers±5 nanometers, said shift leading to a change in said visible color to a violet-blue color; and
d) optionally, including a standardized preparation of said protease that cleaves said peptide substrate, said standardized preparation being useful for preparing standard curves of proteolytic activity of said protease.

4. An assay kit as recited in claim 3, wherein said gold nanoparticles have a size in at least one dimension of from 1 to 200 nanometers and a shape selected from the group consisting of a sphere, a rod, a prism, a cube, a disk, a core-shell structure, a frame, a cage, a mixture thereof.

5. An assay kit as recited in claim 3, wherein said gold nanoparticles are provided as a solution in one of water, methanol, ethanol, acetone, a biological buffer, or a mixture thereof.

6. An assay kit as recited in claim 3, wherein said gold nanoparticles are provided as a nanostructure at least partially covered by said gold nanoparticles.

7. An assay kit as recited in claim 3, wherein said at least one first functional group comprises a thiol group, an amine group, a phosphine group, a disulfide group or a mixture thereof.

8. An assay kit as recited in claim 3, wherein said at least one second functional group comprises arginine, lysine, histidine, aspartic acid, glutamic acid, or a mixture thereof.

9. An assay kit as recited in claim 3, wherein if said surface charge is negative then said at least one second functional group is selected from the group consisting of lysine, arginine, histidine, and mixtures thereof and wherein if said surface charge is positive then said at least one second functional group is selected from the group consisting of aspartic acid, glutamic acid, and mixtures thereof.

10. An assay kit as recited in claim 3, wherein said cleavage site is cleavable by at least one of a serine protease, a threonine protease, a cysteine protease, an aspartate protease, a glutamic acid protease, a metalloprotease, or a mixture thereof.

11. An assay kit as recited in claim 3 wherein said cleavage site is cleavable by at least one of trypsin, chymotrypsin, thrombin, prostrate-specific antigen, HIV-1 protease, elastase, a metalloendopeptidase, subtilisin, or a mixture thereof.

12. An assay kit as recited in claim 3 further comprising said standardized preparation of said protease, said protease capable of cleaving said peptide substrate, wherein said kit is usable to detect the activity of a protease inhibitor of said standardized protease, said protease inhibitor activity measured by monitoring a reduction in the proteolytic activity of said standardized protease when in the presence of said protease inhibitor.

13. An assay kit as recited in claim 12, wherein said protease inhibitor comprises at least one of saquinavir, ritonavir, indinavir, nelfinavir, amprenavir, boceprevir, telaprevir, bovine pancreatic trypsin inhibitor, serine protease inhibitor Kazal-type 1, Alpha-1 antitrypsin, or mixtures thereof.


SEQUENCE LISTING (12 sequences)

SEQ ID NO: 1 - protein, 10 aa, ARTIFICIAL SEQUENCE
  Contains at least one cleavage site for the enzyme Trypsin
KKGFPRGGDC

SEQ ID NO: 2 - protein, 4 aa, ARTIFICIAL SEQUENCE
  one of the cleavage fragments from Seq ID NO 1 after cleavage of SEQ ID NO 1 by Trypsin
GFPR

SEQ ID NO: 3 - protein, 4 aa, ARTIFICIAL SEQUENCE
  One of the hydrolytic fragments produced from SEQ ID NO 1 after cleavage by Trypsin
GGDC

SEQ ID NO: 4 - protein, 10 aa, ARTIFICIAL SEQUENCE
  Contains at least one recognized cleavage site for the enzyme Trypsin
CGFPRGGSDE

SEQ ID NO: 5 - protein, 5 aa, ARTIFICIAL SEQUENCE
  One of the hydroytic fragments produced from SEQ ID NO 4 after cleavage by Trypsin
CGFPR

SEQ ID NO: 6 - protein, 5 aa, ARTIFICIAL SEQUENCE
  One of the hydrolytic fragments produced from SEQ ID NO 4 after cleavage by Trypsin
GESDE

SEQ ID NO: 7 - protein, 6 aa, ARTIFICIAL SEQUENCE
  Contains a published recognition site for the enzyme Thrombin
LVPRGS

SEQ ID NO: 8 - protein, 13 aa, ARTIFICIAL SEQUENCE
  Contains at least one cleavage site for the enzyme Protease K
CGGFSTSLRARKA

SEQ ID NO: 9 - protein, 4 aa, ARTIFICIAL SEQUENCE
  One of the hydrolytic fragments produced from SEQ ID NO 8 after cleavage by Protease K
CGGF

SEQ ID NO: 10 - protein, 12 aa, ARTIFICIAL SEQUENCE
  Contains at least one cleavge site for the enzyme Thrombin
AKRGRGSTSFGC

SEQ ID NO: 11 - protein, 5 aa, ARTIFICIAL SEQUENCE
  One of the hydrolytic fragments produced from SEQ ID NO 10 after cleavage by Thrombin
AKRGR

SEQ ID NO: 12 - protein, 7 aa, ARTIFICIAL SEQUENCE
  One of the hydrolytic fragments produced from SEQ ID NO 10 after cleavage by Thrombin
GSTSFGC